(19) United States
(12) Patent Application Publication
(10) Pub. No.: US 2014/0147838 A1
(43) Pub. Date: May 29, 2014

(54) METHOD FOR MULTICOLOR CODETECTION OF MICRORNA AND PROTEINS

(71) Applicants: Lorenzo F. Sempere, Lebanon, NH (US); Murray Korc, Indianapolis, IN (US)

(72) Inventors: Lorenzo F. Sempere, Lebanon, NH (US); Murray Korc, Indianapolis, IN (US)

(73) Assignee: TRUSTEES OF DARTMOUTH COLLEGE, Hanover, NH (US)

(21) Appl. No.: 13/811,010

(22) PCT Filed: Jun. 16, 2011
(86) PCT No.: PCT/US11/40624

§ 371 Date: Feb. 4, 2013

Related U.S. Application Data
(60) Provisional application No. 61/441,694, filed on Feb. 11, 2011.
(60) Provisional application No. 61/447,882, filed on Mar. 1, 2011.
(60) Provisional application No. 61/377,245, filed on Aug. 26, 2010.
(60) Provisional application No. 61/365,945, filed on Jul. 20, 2010.

(51) Int. Cl. C12Q 1/68

(52) U.S. Cl. CPC C12Q 1/6886
USPC 435/6.11

(57) ABSTRACT

The present invention is directed to systems and methods of conducting a purchase transaction of eligible goods or services using a stored value associated with an indicia. Methods in accordance with the invention may include steps

### INTRODUCTION

This application claims priority to U.S. Provisional Application No. 61/447,882, filed Mar. 1, 2011; U.S. Provisional Application No. 61/441,694, filed Feb. 11, 2011; U.S. Provisional Application No. 61/377,245, filed Aug. 26, 2010; and U.S. Provisional Application No. 61/365,945, filed Jul. 20, 2010, which are incorporated herein by reference.

This invention was made with government support under grant numbers R21CA133715, R21 RR024411-01A1, and R03 CA141564-01 awarded by the National Institutes of Health and National Cancer Institute. The government has certain rights in the invention.

### BACKGROUND OF THE INVENTION

MicroRNAs (miRNAs) are a class of short non-coding regulatory RNA genes, which act as post-transcriptional regulators of gene expression (Lee & Ambros (2001) Science 294(5543):862-4; Lau, et al. (2001) Science 294(5543):858-62; Lagos-Quintana, et al. (2001) Science 294(5543):853-8). By binding to the 3′-untranslated region of target mRNAs, the ˜21-23 nucleotide-long miRNAs can trigger translational downregulation and/or increased degradation of mRNA of target genes (Bartel (2009) Cell 136(2):215-33). The recent explosion of miRNA research in biomedical sciences and particularly in cancer biology attests to their importance to human disease (Ventura & Jacks (2009) Cell 136(4):586-91; Sempere & Kauppinen (2009) In: Handbook of Cell Signaling. 2^{nd} ed. Oxford: Academic Press, Bradshaw & Dennis (eds.) pg. 2965-81). High-throughput expression profiling of RNA extracted from whole tissue biopsies has provided short lists of miRNAs that could serve as useful biomarkers for early detection, diagnosis and/or prognosis of different types of cancer (Barbarotto, et al. (2008) Int. J. Cancer 122(5):969-77). Low levels of let-7, miR-34, miR-126, miR-145 and high levels of miR-21, miR-155, miR-221 have been frequently reported in association with breast, colorectal, gastrointestinal, lung, pancreas, prostate and/or thyroid cancer (Barbarotto, et al. (2008) supra; Volinia, et al. (2006) Proc. Natl. Acad. Sci. USA 103(7):2257-61). These high-throughput profiling results have been technically confirmed by miRNA-specific quantitative RT-PCR analysis and several studies based on RT-PCR analysis of miRNA expression have further supported the clinical application of miRNAs as informative biomarkers. However, these detection assays cannot directly determine whether these expression changes occur specifically within cancer cells, reactive stromal and/or infiltrating immune cells recruited to the cancerous lesion. Moreover, tissue heterogeneity among specimens and unequal representation of source cells (cancer cell and/or other cell types) with altered miRNA expression may confound interpretation of RT-PCR analyses, unless they are performed on samples highly-enriched for the source cells. Visualization of miRNA expression within individual cells by in situ hybridization (ISH) is needed to provide an independent tool to clinically validate miRNAs that have been highlighted by expression profiling analysis and also to more closely assess the etiological relevance and clinical significance of altered miRNA expression.

Locked nucleic acids (LNAs), a class of bicyclical high-affinity RNA analogues (Kauppinen, et al. (2006) Handb. Exp. Pharmacol. 173:405-22) have been shown to provide specific and avid hybridization to the short RNA sequence of mature miRNAs in zebrafish and mouse embryos by whole-mount ISH using chromogenic staining (Kloosterman, et al. (2006) Nat. Methods 3(1):27-9; Wienholds, et al. (2005) Science 309(5732):310-1). Subsequently, ISH methods have been implemented to detect miRNA expression in formalin-fixed paraffin-embedded (FFPE) brain (Nelson, et al. (2006) RNA 12(2):187-91), breast (Sempere, et al. (2007) Cancer Res. 67(24):11612-20), colon (Yamamichi, et al. (2009) Clin. Cancer Res. 15(12):4009-16), lung (Liu, et al. (2010) J. Clin. Invest. 120(4):1298-309), and pancreatic tissue sections (Dillhoff, et al. (2008) J. Gastrointest. Surg. 12(12):2171-6; Habbe, et al. (2009) Cancer Biol. Ther. 8(4):340-6). These methods followed a similar general strategy in which DNA probes modified with LNAs were terminally-tagged with a hapten molecule either digoxigenin (DIG) or fluorescein (FITC or FAM), and a single step antibody conjugated to alkaline phosphatase (AP) or horseradish peroxidase (HRP) was used for probe recognition and signal staining. AP-mediated (i.e., BCIP/NBT) or HRP-mediated (i.e., DAB) chromogenic staining of tissue sections (Nelson, et al. (2006) supra; Yamamichi, et al. (2009) supra; Dillhoff, et al. (2008) supra; Habbe, et al. (2009) supra) has been used, as has HRP-mediated (i.e., FITC) fluorescent staining (Sempere, et al. (2007) supra; Liu, et al. (2010) supra). These recent advancements showed the feasibility of detecting cancer-associated miRNAs by ISH in FFPE tissue specimens, but further technical improvements in signal quantification, reproducibility and sensitivity were still required for the development of miRNA-based clinical assays.

A method for the simultaneous codetection of isolated DNA and proteins under identical conditions, notably in the same buffer and at the same temperature, has been suggested (Perrin, et al. (2003) Anal. Biochem. 322:148-155). However, this assay uses a single detection tool, alkaline phosphatase and the BM-purple-AP substrate. Therefore, this assay is not suitable for the codetection/localization of DNA and proteins in a single FFPE tissue sample. Moreover, while methods for the codetection of multiple targets such as non-coding RNAs and proteins using different labels have been described (de Planell-Saguer, et al. (2010) Nat. Protocols 5:1061-1073; US 2009/0156428), these methods do not contemplate removing or destroying probes or labels between the detection of the multiple targets. Therefore, interference from the reagents used in previous detection steps can decrease the sensitivity of these methods and limit the number of independent markers that can be codetected.

### SUMMARY OF THE INVENTION

The present invention features methods and a kit for multicolor codetection of miRNA and proteins for use in diagnostic and prognostic application. In one embodiment, the method of the invention involves the steps of (a) contacting a biological sample with a probe that binds to a miRNA, (b) detecting binding of the probe to the miRNA with a label, (c) inactivating the label, (d) contacting the biological sample with a binding agent that specifically binds to a protein, and (e) detecting binding of the binding agent to the protein, thereby codetecting the miRNA and protein in the biological sample. In one embodiment, the label is composed of at least one hapten, an anti-hapten antibody, and an enzyme-conjugated secondary antibody specific for the anti-hapten antibody. In accordance with this embodiment, the enzyme of the enzyme-conjugated secondary antibody is horseradish peroxidase. In other embodiments, the probe is selected from the group of SEQ ID NO:1 to 56 and is a modified probe. In further embodiments, the binding agent that specifically binds to the protein comprises an antibody and binding of the binding agent to the protein is detected by (i) contacting the antibody with an enzyme-conjugated secondary antibody, and (ii) detecting the enzyme activity, e.g., horseradish peroxidase activity. In still other embodiments, the biological sample is a biopsy sample and the protein is selected from the group of amylase, cytokeratin (CK) 5/6, CK7, CK8/18, CK14, CK19, CK20; cluster of differentiation (CD) 3, CD4, CD8, CD11b, CD11c, CD19, CD20, CD31, CD34, CD24, CD44, CD45, CD68, CD86, CD105; myeloperoxidase; E-cadherin; laminin; estrogen receptor (ER); Glucagon; Human Epidermal growth factor Receptor 2 (HER2); Insulin; Ki-67; phosphorylated v-akt murine thymoma viral oncogene (pAKT); protein 15 (p15), p16, p21, p27, p53, p63; mutS homolog 6 (MSH-6); Proliferating Cell Nuclear Antigen (PCNA); Progesterone Receptor (PR); Phosphatase and Tensin Homolog (PTEN); Smooth Muscle Actin; Tubulin; Vimentin; Cyclin D1; Cyclin E and somatostatin.

In another embodiment, the method of the invention involves the steps of (a) contacting a biological sample with a probe that binds to a miRNA, (b) detecting binding of the probe to the miRNA with a label, (c) inactivating the label, (d) contacting the biological sample with an antibody that specifically binds to a protein, (e) detecting binding of the antibody to the protein with a secondary antibody reagent, (f) inactivating the secondary antibody reagent, and (g) repeating steps (a) to (f) for the codetection of multiple miRNAs and proteins in the biological sample.

In accordance with the present invention, a kit is composed of a probe that binds to a miRNA, and a binding agent that specifically binds to a protein, wherein the labeled probe is selected from the group of SEQ ID NO:1 to 56.

### BRIEF DESCRIPTION OF THE DRAWINGS

FIG. 1 shows the reproducibility of the ISH method and signal analysis. Matched normal and tumor FFPE breast tissue sections were used to codetect miR-205 and U6 snRNA using FAM2X and Bio2X-tagged probes, respectively. miR-205 and U6 signal was revealed by sequential TSA reactions with FITC-tyramine (green for miR-205 probe) and Rhodamine-tyramine (red for U6 probe) substrates. Consecutive (i, iii) matched normal (N) and tumor (T) tissue sections were assayed on separate days (1 and 2). Raw fluorescent images of the same representative field for each intra-experimental replica and a heat map rendition of intensity classes of miR-205 signal above background noise were generated. FIG. 1A shows the signal intensity of miR-205 measured across the mammary duct in N1 tissue and across invasive carcinoma lesion in T1 (upper panel). Line profile of N1iii was slightly shifted to the right (gap) to match the signal from myoepithelial (myo) and luminal (lu) cellular structures of N1i and N1ii. Distribution of pixel intensity of the whole images indicates concordant readings for each intra- and inter-experimental replica (mid panel). Bar graph displays means and standard deviations of percentage of pixels within each intensity class for each set of intra-experimental replica (lower panel). Raw images of miR-205 and snRNA U6 were captured with the same exposure and gain setting in normal and tumor tissue were displayed as a heat map graph. FIG. 1B shows the signal intensity of miR-205 and U6 expression. Background intensity was subtracted from the recorded intensity value and these corrected intensity values were used to generate line graphs. Average intensity of miR-205 and U6 signal of selected structures was used to calculate relative fold decrease of miR-205 expression in luminal and cancer cells compared to myoepithelial cells (lu/myo and ICa/myo, respectively).

FIG. 2 shows the codetection of miRNAs with cell-type specific and prognostic protein markers. Serial FFPE tissue sections of the indicated organs were stained with H&E to reveal histological features or subject to ISH assay using FAM2X-tagged probes against miR-34a, miR-126, miR-141, miR-214 and miR-375 mixed with Bio2X-tagged probe against 18S rRNA, BrdU2X-tagged probe against miR-205 or DIG2X-tagged probe against let-7a. miRNA signal was revealed by sequential TSA reactions with FITC-tyramine, Rhodamine-tyramine and AMCA-tyramine substrates. After heat-induced epitope retrieval (HIER) as needed, expression of indicated proteins was revealed by sequential TSA reactions with DYLIGHT594-tyramine or DYLIGHT680-tyramine and AMCA-tyramine substrates, each TSA reaction was preceded by incubation with specific antibodies against each protein and appropriate anti-source species/HRP antibody; except for insulin expression, which was revealed by an anti-guinea pig secondary antibody conjugated to CY3 obviating the need for the TSA step. Line profile analysis was used to quantitate intensity of RNA or protein expression. Background intensity was subtracted and intensity values were normalized setting the point with maximum intensity to 100 and calculating other values in relation to this reference. miRNA expression pattern was plotted as a line; independently, expression patterns of each protein were plotted as stacked areas.

FIG. 3 shows the co-localization of miR-155 with CD45 immune cell marker. Serial 4-μm FFPE tumor tissue sections of the indicated organs were stained with H&E to reveal histological features or subject to standard ISH assay using FAM2X-tagged probe against miR-155. MiR-155 signal was revealed by TSA reaction with FITC-tyramine. CK19 expression was revealed by TSA reaction with DYLIGHT680-tyramine. After a HIER with citrate, CD45 expression was revealed by TSA reaction with Rhodamine-tyramine. Tissue sections were counterstained with nuclear marker DAPI. Line profile analysis was used to quantify intensity of RNA or protein expression. Background intensity was subtracted and intensity values were normalized setting the point with maximum intensity to 100 and calculating other values in relation to this reference. miRNA expression pattern was plotted as a line; independently, expression patterns of each protein were plotted as stacked areas.

FIG. 4 shows the codetection of miR-21 with PTEN in clinical specimens. Serial 4-μm FFPE tumor tissue sections of the indicated organs were stained with H&E to reveal histological features or subject to standard ISH assay using FAM2X-tagged probe against miR-21 and Bio2X-tagged probe against snRNA U6. MiR-21 and snRNA U6 signal was revealed by sequential TSA reactions with FITC-tyramine and AMCA-tyramine substrates. Smooth muscle actin (SMA) expression was revealed by TSA reaction with DYLIGHT594. After a HIER with citrate, PTEN and Vimentin (VIM) expression was revealed by TSA reaction with Rhodamine-tyramine and DYLIGHT680, respectively. Line profile analysis was used to quantitate intensity of miR-21, PTEN and VIM expression. Background intensity was subtracted and intensity values were normalized setting the point with maximum intensity to 100 and calculating other values in relation to this reference. VIM expression pattern was plotted as a line; independently, relative expression of miR-21 and PTEN for each data point was plotted as stacked areas.

FIG. 5 is a schematic representation of an illustrative multi-color ISH/IHC assay. A FFPE tumor tissue section is subject to standard ISH assay using FAM2X-tagged probe against a miRNA and Bio2X-tagged probe against snRNA U6. The miRNA and snRNA U6 signal is revealed by sequential TSA reactions with FITC-tyramine (color 1) and AMCA-tyramine (color 2) substrates. Immediately after expression of a protein marker such epithelial-specific cytokeratin 19 (CK19) is revealed by TSA reaction with DYLIGHT680 (color 3). After a heat-induced epitope retrieval with citrate (which is required for several protein markers). Expression of immune cell-specific (CD45) and mesenchymal cell-specific (SMA) marker is revealed by TSA reaction with Rhodamine-tyramine (color 4) and DYLIGHT594 (color 5).

### DETAILED DESCRIPTION OF THE INVENTION

Examination of formalin-fixed paraffin embedded (FFPE) tissues is the cornerstone for histological and molecular pathology diagnosis of solid tumors. High-throughput profiling experiments have linked altered expression of miRNAs to different types of cancer. Several methodologies have been applied to detect miRNAs in FFPE specimens. However, tumor tissues are a heterogeneous mixture of not only cancer cells, but also supportive and reactive tumor microenvironment elements. In this respect, in situ hybridization (ISH) assays have the unique feature of determining the cellular compartment(s) of altered miRNA expression, which is demonstrated herein as being paramount to accurately interpreting the clinical significance of miRNA changes. The instant analysis describes a combination of ISH and IHC assays to study a subset of cancer-associated miRNAs, including frequently down-regulated (e.g., miR-34a and miR-126) and up-regulated (e.g., miR-21, miR-155 and miR-10b) miRNAs, in a variety of cancers including breast, colorectal, lung, pancreas and prostate carcinomas. Despite the distinct histopathological alterations of each particular cancer type, general trends emerged that pinpointed to distinct source cells of altered miRNA expression. While altered expression of miR-21 and miR-34a was manifested within cancer cells, that of miR-126 and miR-155 was predominantly confined to endothelial cells and immune cells, respectively. Moreover, miR-10b levels were highest in pancreatic ductal adenocarcinoma, intermediate in intraductal papillary mucinous neoplasms, and lowest in benign cysts These results indicate a heterogeneous participation of miRNAs in carcinogenesis by intrinsically affecting cancer cell biology or by modulating stromal, vascular and immune responses. Accordingly, implementation of ISH in combination with IHC for the detection of miRNAs and clinically important protein markers in FFPE specimens now provides a fluorescence-based multi-color ISH/IHC assay that is rapid, sensitive, compatible with current automated clinical IHC assays, and provides spatial characterization of miRNA expression.

In this respect, the present invention features a method for the codetection of miRNA and protein in a biological sample by (a) contacting a biological sample with a probe that binds to a miRNA, (b) detecting binding of the probe to the miRNA with a label, (c) inactivating the label, (d) contacting the biological sample with a binding agent that specifically binds to a protein, and (e) detecting binding of the binding agent to the protein, thereby codetecting the miRNA and protein in the biological sample. By detection is meant detection in the sense of presence versus absence of one or more miRNAs and/or proteins; the registration of the level or degree of expression of one or more miRNAs and/or proteins; and/or the localization of one or more miRNAs and/or proteins at the tissue, cellular or subcellular level.

A biological sample as defined herein is a small part of an individual, representative of the whole and may be constituted by a biopsy or a body fluid sample. Biopsies are small pieces of tissue and may be fresh, frozen or fixed, such as formalin-fixed and paraffin embedded (FFPE). Body fluid samples may be blood, plasma, serum, urine, sputum, cerebrospinal fluid, milk, or ductal fluid samples and may likewise be fresh, frozen or fixed. Samples may be removed surgically, by extraction, i.e., by hypodermic or other types of needles, by microdissection or laser capture. The sample may be any sample as described herein and in certain embodiments is a FFPE sample.

In this respect, particular embodiments of the instant method encompass in situ detection. The term in situ method refers to the detection of miRNA and protein in a sample wherein the structure of the sample has been preserved. This may thus be a biopsy wherein the structure of the tissue is preserved. In situ methods are generally histological, i.e., microscopic in nature and include but are not limited to methods such as in situ hybridization techniques and immunohistochemical methods. In situ hybridization (ISH) applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH is used to assay expression and gene expression patterns in a tissue/across cells, such as the expression of miRNAs/nucleic acid molecules as described herein.

As is apparent from the instant data, the method of this invention is of particular use in the detection of miRNA and proteins in the detection, classification, diagnosis or prognosis of hyperproliferative diseases especially cancers and specifically breast, colorectal, gastrointestinal, lung, pancreas, prostate and/or thyroid cancers. Accordingly, in particular embodiments, the biological sample is a biopsy sample or a body fluid containing tumor and/or tumor-associated tissue or cells. When the biological sample is for the detection of a hyperproliferative disease, it may be desirable to obtain more than one sample, such as two samples, such as three samples, four samples or more from individuals, and preferably the same individual. The at least two samples may be taken from normal tissue and hyperproliferative tissue, respectively. This allows the relative comparison of expression both as in the presence, absence, level of expression, and/or localization of at least one miRNA and protein between the two samples. Alternatively, a single sample may be compared against a “standardized” sample, such a sample containing material or data from several samples, preferably also from several individuals. A standardized sample may include either normal or hyperproliferative sample material or data.

As used herein, the terms “cancer,” “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as nonpathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of nonpathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal cell carcinoma, prostate cancer, pancreatic and/or testicular tumors, non-small cell carcinoma of the lung, cancer of the small intestine and cancer of the esophagus.

As is conventional in the art, a miRNA or microRNA refers to a 19-25 nt non-coding RNA derived from an endogenous gene that acts as a post-transcriptional regulator of gene expression. MiRNAs are processed from longer (ca 70-80 nt) hairpin-like precursors termed pre-miRNAs by the RNAse III enzyme Dicer. MiRNAs assemble in ribonucleoprotein complexes termed miRNPs and recognize their target sites by antisense complementarity thereby mediating down-regulation of their target genes. Near-perfect or perfect complementarity between the miRNA and its target site results in target mRNA cleavage, whereas limited complementarity between the miRNA and the target site results in translational inhibition of the target gene. Using the method of this invention, the presence, absence, level or localization of any miRNA can be determined. In particular embodiments, the presence, absence, level or localization of a miRNA of the invention is associated with a hyperproliferative disease, especially cancer, and specifically breast, colorectal, gastrointestinal, lung, pancreas, prostate and/or thyroid cancer. Such miRNAs include, but are not limited to, those miRNAs exemplified herein (see Table 1), as well as miRNA including miR 196b (HoxA9), 10b, p14, 328, 30A-3P, 125b5, 30E-3P, 680, 134, 604, 128b, 128a, 331, 520F, 299-3P, 520H, 510, 365, 520G, 9, 324-3P, 351, 125A, 146a, 764-5P, 302D, 520D, 652, 520C, 350, 585, 621, 542-5P, 560, 126, and 341. As will be readily appreciated by the skilled artisan, the instant method can be adapted to the detection of virtually any non-coding RNA including small nuclear (snRNAs), ribosomal RNAs (rRNAs), transfer RNAs (tRNAs) ultraconserved genetic elements, Piwi-interacting RNAs (piRNA), small interfering RNAs (siRNA), and mirtrons.

In this respect, the present invention further includes the detection of other non-coding RNAs such as, but not limited to, snRNA (e.g., U6), or rRNA (e.g., 18S) in addition to the detection of a miRNA and protein marker. These non-coding RNAs are of use in facilitating the quantification of changes in miRNA expression, assessing quality of the biological sample, and serving as an internal control during sample processing and analysis. Moreover, codetection of other non-coding RNAs and miRNAs may serve to investigate regulatory interactions among these species, or provide for contextualization of the changes of miRNA expression or the other non-coding RNA. A ratio of expression changes of these non-coding RNA species would be more informative than individual detection in independent samples.

To detect the presence, absence, level or localization of miRNA, the method of the invention employs a probe. The term “probe” refers to a defined oligonucleotide or a nucleic acid molecule used to detect a target miRNA nucleic acid molecule by hybridization, in particular in situ hybridization. In this respect, a probe bears a complementary sequence to the target miRNA. Probes of the invention can be single-stranded DNA, double-stranded DNA, RNA or a combination of DNA and RNA. In some embodiments, the probe of the invention is synthesized or produced with conventional oligonucleotides. In other embodiments, the probe is modified to include chemical modifications.

If present, chemical modifications of a probe can include, singly or in any combination, 2′-position sugar modifications, 5-position pyrimidine modifications (e.g., 5-(N-benzylcarboxyamide)-2′-deoxyuridine, 5-(N-isobutylcarboxyamide)-2′-deoxyuridine, 5-(N-tryptaminocarboxyamide)-2′-deoxyuridine, 5-(N-[1-(3-trimethylammonium) propyl]carboxyamide)-2′-deoxyuridine chloride, 5-(N-napthylmethylcarboxyamide)-2′-deoxyuridine, 5-(Imidazolylethyl)-2′-deoxyuridine, and 5-(N-[1-(2,3-dihydroxypropyl)]carboxyamide)-2′-deoxyuridine), 8-position purine modifications, modifications at exocyclic amines, substitution of 4-thiouridine, substitution of 5-bromo- or 5-iodo-uracil, backbone modifications, methylations, unusual base-pairing combinations such as the isobases isocytidine and isoguanidine, and the like.

In one embodiment, 5-position pyrimidine modifications refer to pyrimidines with a modification at the C-5 position. Examples of a C-5 modified pyrimidine include those described in U.S. Pat. Nos. 5,719,273 and 5,945,527. Examples of a C-5 modification include substitution of deoxyuridine at the C-5 position with a substituent selected from benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), and isobutylcarboxyamide (alternatively isobutylaminocarbonyl) (iBu) In this respect, representative C-5 modified pyrimidines include 5-(N-benzylcarboxyamide)-2′-deoxyuridine (BndU), 5-(N-isobutylcarboxyamide)-2′-deoxyuridine (iBudU), 5-(N-tryptaminocarboxyamide)-2′-deoxyuridine (TrpdU) and 5-(N-napthylmethylcarboxyamide)-2′-deoxyuridine (NapdU).

Modified probes of the invention include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). In particular embodiments, a modified probe of the invention contains at least one nucleoside analog, e.g., a locked nucleic acid (LNA). The synthesis and preparation of LNA monomers adenine, cytosine, guanine, 5-methyl-cytosine, thymine and uracil, along with their oligomerization, and nucleic acid recognition properties have been described (Koshkin, et al. (1998) Tetrahedron 54:3607-3630). LNAs and preparation thereof are also described in WO 98/39352 and WO 99/14226.

Other modifications include 3′ and 5′ modifications, such as capping. Further, any of the hydroxyl groups ordinarily present in a sugar may be replaced by a phosphonate group or a phosphate group; protected by any suitable protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, or organic capping group moieties of from about 1 to about 20 polyethylene glycol (PEG) polymers or other hydrophilic or hydrophobic biological or synthetic polymers.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

As noted above, one or more phosphodiester linkages may be replaced by alternative linking groups. These alternative linking groups include embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (C1-C20) optionally containing an ether (—O—) linkage, aryl, alkenyl, cycloalkyl, cycloalkenyl or araldyl. Not all linkages in a probe need be identical. Substitution of analogous forms of sugars, purines, and pyrimidines can be advantageous in designing a final product, as can alternative backbone structures like a polyamide backbone, for example.

If present, a modification to the nucleotide structure of a probe may be imparted before or after assembly of the probe. A sequence of nucleotides may be interrupted by non-nucleotide components. A probe may be further modified after polymerization, such as by conjugation with a labeling component.

In particular embodiments, the probe has a nucleotide sequence selected from the group of SEQ ID NOs: 1 to 56, or a fragment thereof that can hybridize under stringent condition, and/or has an identity of at least 80% to any of these sequences. In particular embodiments the probe is selected from the group of SEQ ID NOs: 2, 5, 8, 9, 10, 13, 16, and 18, or a fragment thereof that can hybridize under stringent conditions, and/or has an identity of at least 80% to any of these sequences.

In accordance with the present method, a probe is detected with a label or tag or otherwise modified to facilitate detection. A label or a tag is an entity making it possible to identify a compound to which it is associated. It is within the scope of the present invention to employ probes that are labeled or tagged by any means known in the art such as, but not limited to, radioactive labeling, affinity labeling (e.g., with a hapten and its associated antibody), fluorescent labeling and enzymatic labeling. Furthermore, the probe may be immobilized to facilitate detection. In particular embodiments, a hapten is incorporated into the probe of the invention. Haptens commonly employed in labeling applications include fluorescein (e.g., 5- or 6-carboxy-fluorescein, FAM), biotin, digoxigenin (DIG), 5-bromo-2-deoxyuridine (BrdU) and dinitrophenol. Probe synthesis and hapten incorporation are routinely practiced in the art and any suitable method can be employed. See, e.g., Luehrsen, et al. (2000) J. Histochem. Cytochem. 48:133-145.

Detection of probes with labels as described herein is routinely practiced in the art and any suitable method can be employed. In particular embodiments, the probe contains a hapten that is detectable using an immunoassay. Accordingly, certain embodiments of this invention include the use of an anti-hapten antibody. In this respect, binding of the probe to the miRNA can be detected by contacting the hapten with an anti-hapten antibody, contacting the anti-hapten antibody with a secondary antibody reagent, and detecting the secondary antibody reagent by routine methods as described herein. For the purposes of the present invention, a secondary antibody reagent is composed of an antibody covalently linked to a protein that provides for a detectable signal. Suitable detectable proteins include, but are not limited to, fluorescent proteins, chromogenic proteins, and enzymes that catalyze the production of a product that is luminescent, fluorescent, or colored (e.g., β-galactosidase, luciferase, horse radish peroxidase, alkaline phosphatase, etc.). Suitable fluorescent proteins include, but are not limited to, green fluorescent protein (GFP; Chalfie, et al. (1994) Science 263(5148):802-805); enhanced GFP (EGFP; Clontech Laboratories, Inc.); blue fluorescent protein (BFP; Stauber (1998) Biotechniques 24(3):462-471; Heim & Tsien (1996) Curr. Biol. 6:178-182); enhanced yellow fluorescent protein (EYFP; Clontech Laboratories, Inc.); and the like. Secondary antibodies linked to various enzymes (i.e., enzyme-conjugated) are commercially available from, for example, Sigma and Amersham Life Sciences (Arlington Heights, Ill.). In certain embodiments, horseradish peroxidase-conjugated secondary antibodies are used in the detection steps of the instant method. In particular embodiments, the invention embraces the use of horseradish peroxidase-mediated tyramide signal amplification (TSA) to enhance detection.

As is known in the art, there are a variety of luminescent, fluorescent, or colored substrates for detecting the activity of enzyme-conjugated secondary antibodies, e.g., by microscopy. For example, horseradish peroxidase-labeled secondary antibody is readily detected with 3,3′-Diaminobenzidine (DAB) and as alkaline phosphatase labeled secondary antibody is readily detected with 5-bromo-4-chloro-3′-indolylphosphate p-toluidine salt (BCIP) and nitro-blue tetrazolium chloride (NBT), both of which are commercially available from a variety of sources (e.g., Pierce Chemical Co., Rockford, Ill.). The enzymatic reaction forms an insoluble colored product wherever antigen-antibody complexes occur.

To increase the number of markers detected in one sample, certain embodiments of the present invention embrace the use of species and/or isotype-specific secondary antibodies. By way of illustration, the detection of one marker can be achieved with an anti-mouse secondary antibody, whereas the detection of a second marker can be achieved with an anti-rabbit secondary antibody. Alternatively, but not mutually exclusive, the detection of one marker can be achieved with an anti-IgG1-specific mouse secondary antibody, whereas the detection of a second marker can be achieved with an anti-IgG2a specific secondary antibody.

To increase the sensitivity of detecting enzyme-conjugated secondary antibodies, assays based on fluorescence or luminescence are typically employed. Fluorescent compounds containing fluorophores, also known as fluorochromes, have the ability to absorb energy from incident light and emit the energy as light of a longer wavelength and lower energy. Fluorescein and rhodamine, usually in the form of isothiocyanates that can be readily coupled to antigens and antibodies, are most commonly used in the art (Stites, et al. (1994) Basic and Clinical Immunology). Fluorescein absorbs light of 490 nm to 495 nm in wavelength and emits light at 520 nm in wavelength. Tetramethylrhodamine absorbs light of 550 nm in wavelength and emits light at 580 nm in wavelength. Illustrative fluorescence-based substrates are described herein and further include such examples as ELF-97 alkaline phosphatase substrate (Molecular Probes, Inc., Eugene, Oreg.); PBXL-1 and PBXL-3 (phycobilisomes conjugated to streptavidin); and CY substrates. ELF-97 is a nonfluorescent chemical that is digested by alkaline phosphatase to form a fluorescent molecule. Because of turnover of the alkaline phosphatase, use of the ELF-97 substrate results in signal amplification. Illustrative luminescence-based detection reagents include CSPD and CDP star alkaline phosphatase substrates (Roche Molecular Biochemicals, Indianapolis, Ind.); and SUPERSIGNAL horseradish peroxidase substrate (Pierce Chemical Co., Rockford, Ill.).

In addition to miRNA, the present method also features the codetection of a protein in the biological sample. Using the method of this invention, the presence, absence, level or localization of any protein can be determined. Proteins that can be detected in accordance with the present invention include, but are not limited to, those proteins exemplified herein (see Table 3), as well as other proteins including COX-2, p16, CD73, CD138, notch receptor-3, CD90, BMI-1, IGF2, YKL-40, EGF-R, c-jun, PCNA, JNK, cyclin B1, c-kit, STAT3, cyclin D1, PI3K, MAPK, MAPKK, DDR2, TRF2, activin, EGFR, HER-2, HER-3, HER-4, MEK1/2 and/or phosphorylated or post-translationally modified versions of said proteins, e.g., phosphorylated EGFR (pEGFR), pHER2, and pHER3. In particular embodiments, the protein being detected is a cell type-specific protein marker or functional marker, i.e., a protein that distinguishes one cell type from another. For example, Ki-67 is a known marker for proliferative cells, CK19 is a known epithelial marker for cancer cells (of epithelial origin; carcinomas), CK7 and CK20 are known differential markers of lung and colorectal cancer cells (of epithelial origin), CD31 is an endothelial marker, and CK5/6 has been shown to be a reliable marker for mesothelioma and squamous cell carcinoma of the lung as well as a marker for the aggressive basal (ER-PR-HER2-; triple negative) subtype of breast carcinoma. Other cell-type specific protein markers are well-known in the art and can be detected in accordance with the present method. As is routine in the art, a protein can be readily detected by a binding agent such as ligand or antibody that specifically binds the protein of interest with little to no detectable binding to any other protein in a sample. In particular embodiments, the binding agent is an antibody specific for the protein of interest. Antibodies to the proteins disclosed herein are well-known in the art and available from a number of commercial sources.

The detection of binding of the binding agent to a protein of interest is routinely practiced in the art and any suitable method can be employed. For example, where the binding agent is a ligand of the protein of interest, said ligand can be labeled or tagged as described herein and detected via an antibody. In embodiments wherein the binding agent is an antibody (i.e., a primary antibody), a secondary antibody reagent (e.g., an enzyme-conjugated secondary antibody) can be used as described herein. In accordance with particular embodiments, binding of the primary antibody to the protein of interest is detected by contacting the primary antibody with an enzyme-conjugated secondary antibody, and detecting enzyme activity by routine methods as described herein. Exemplary enzyme-conjugated antibodies of use in the claimed method include, but are not limited to, horseradish peroxidase-conjugated antibodies and alkaline phosphatase-conjugated antibodies.

Wherein the epitope(s) of the protein of interest are not readily detectable, certain embodiments of this invention further include the use of epitope retrieval methods to facilitate detection of the protein of interest. Such epitope retrieval methods include, but are not limited to, heat-induced epitope retrieval with or the use of commercially available reagents, e.g., Reveal (Biocare Medical) or other pH buffered reagents.

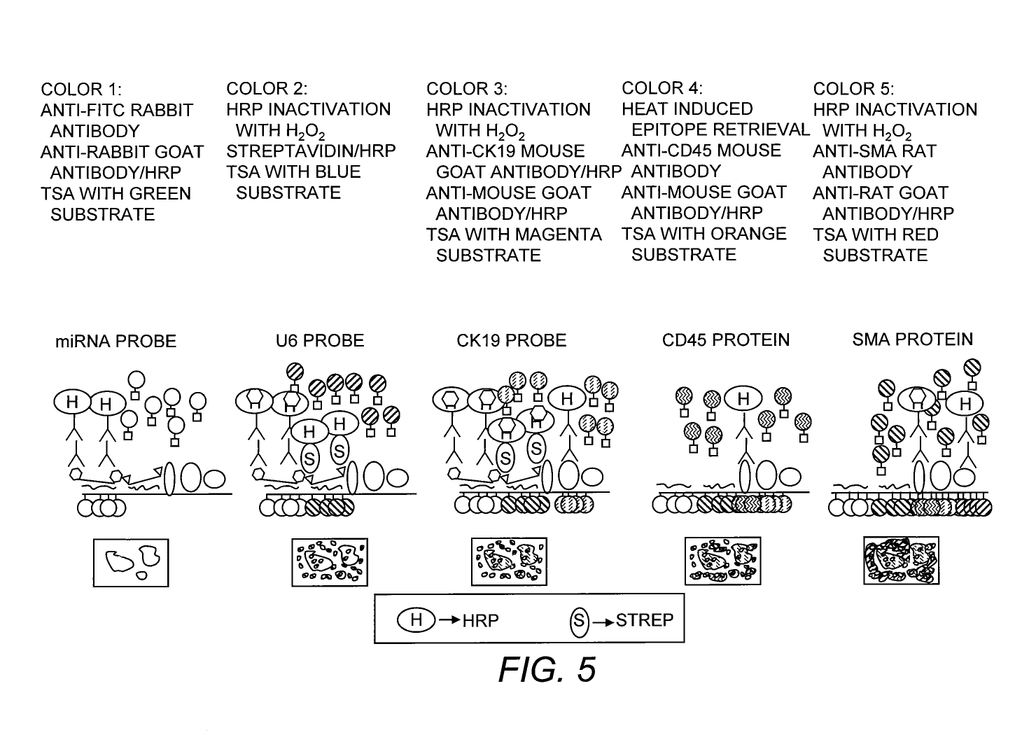

As is readily apparent from the results presented herein, the instant method finds application in the detection of multiple miRNAs and proteins by sequential rounds of detecting individual miRNAs and proteins with different fluorescent substrates (see, e.g., FIG. 5). To facilitate the detection of each individual miRNA and protein without interference from the reagents used in previous detection steps, the present method includes inactivation of the label of the probe and, in some embodiments, inactivation of the secondary antibody reagent. For example, wherein the label of the probe is covalently attached to the probe, e.g., via a protease cleavable linker, said linker can be cleaved to remove the label from the probe thereby inactivating the label. In embodiments wherein the label is a hapten and its associated anti-hapten antibody and enzyme-conjugated secondary antibody specific for the anti-hapten antibody, the label can be rendered inactive by destroying the activity of the enzyme. By way of illustration, sodium azide or hydrogen peroxide is commonly used to block horseradish peroxidase activity and can be used in accordance with the instant method. Likewise, when using a secondary antibody reagent, such as horseradish peroxidase-conjugated secondary antibody, in the detection of a protein of interest, said secondary antibody can likewise be inactivated by sodium azide or hydrogen peroxide.

Accordingly, when detecting multiple miRNAs and proteins in a biological sample, the method of the invention is modified to include the steps of (a) contacting a biological sample with a probe that binds to a miRNA, (b) detecting binding of the probe to the miRNA with a label, (c) inactivating the label, (d) contacting the biological sample with an antibody that specifically binds to a protein, (e) detecting binding of the antibody to the protein with a secondary antibody reagent, (f) inactivating the secondary antibody reagent, and (g) repeating steps (a) to (f) until each of the miRNA and protein of interest have been detected. In embodiments where the miRNA and protein are both detected via the use of enzyme-conjugated secondary antibodies, which can be inactivated between each detection step, it will be readily appreciated by the skilled artisan that there is no particular order in which the miRNA and protein are detected; the miRNA can be detected first or the protein can be detected first. In addition, to facilitate the codetection of the presence, absence, level or localization of miRNA and protein, particular embodiments embrace the use of enzyme substrates with different fluorophores.

For carrying out the instant method, the present invention also features a kit, container, pack, or dispenser containing probes and binding agents of the invention together with instructions for use. The kit may be for the detection of a miRNA and protein, or for the classification, diagnosis and/or prognosis of a disease related to the miRNA and/or protein such as a hyperproliferative disease, e.g. a cancer.

The kit can include the probes and binding agents of the invention in a form suitable for the detection of said miRNA and protein. In this respect, the kit may include any reagent for the detection of said probes or binding agents, e.g., enzyme-conjugated secondary antibodies and substrates. In particular embodiments, the kit includes a probe selected from the group of SEQ ID NO:1 to 56. In specific embodiments the probe is selected from the group of SEQ ID NOs: 2, 5, 8, 9, 10, 13, 16, and 18. Moreover, in particular embodiments, the kit also includes instructions for hybridizing the probe to the miRNA and the binding agent to the protein. Instructions can include a detailed Or step-by-step protocol, including hybridization conditions, to perform the method of the invention. The instructions can be provided as a text file or printout, or when the kit is compatible with an automated staining system, the instructions can be provided as computer file containing the protocol program.

As described herein, sequential rounds of horseradish peroxidase-mediated tyramide signal amplification with different synthesized fluorescent substrates enables codetection of miRNAs, abundant non-coding RNAs and/or proteins for signal quantification, cell type co-localization studies and assessment of miRNA:target interactions. Increased specificity and sensitivity of signal needed to perform ISH with miRNA probes was achieved by designing fluorescein (FITC)-labeled LNA-modified DNA oligonucleotides (with two terminal hapten moieties, e.g., 5′ and 3′ FITC-labeled probes) against the full length of the miRNA (20 to 24 nts) with a melting temperature (Tₘ) between 70-75° C. Furthermore, tyramide signal amplification (TSA) reaction, in which horseradish peroxidase (HRP) was conjugated to an anti-FITC antibody (which binds to the FITC-labeled miRNA probe) activated the tyramide moiety of a fluorescent substrate resulting in a covalent attachment to proteins in the vicinity of the miRNA probe. Using this methodology several miRNA or other RNA species and several protein markers can be codetected on the same tissue section using different fluorescent substrates using a fully-automated staining station. These experiments demonstrated the operator-free capability of this methodology. Intra- and inter-experimental reproducibility of this ISH/IHC assay was assessed. Several examples of codetection of miRNAs and clinically relevant protein markers in a panel of breast, colorectal, lung, pancreatic and prostatic tumors demonstrated the general clinical application of this assay in common types of solid tumors. Thus, this robust and reproducible ISH/IHC assay could be readily adopted in a clinical setting for miRNA-based investigational, diagnostic and prognostic purposes.

Given that multiple miRNAs and proteins can be codetected in a single biological sample with a method that is compatible and meets the reproducibility standards of current clinical IHC assays, the instant method has many clinical applications for disease management. Specifically, using the combined ISH/IHC assay, the expression of a panel of cancer-associated miRNAs in archived FFPE specimens of breast, colorectal, lung, pancreatic and prostate carcinomas were analyzed. The results uncovered a complex and distinct contribution of different cell types to altered expression of individual miRNAs in tumor tissues. For example, the observations herein indicate that altered miRNA expression may not be confined to cancer cells to be etiologically relevant (e.g., miR-21 in tumor associated fibroblasts), whereas other miRNA expression changes may simply reflect tissue heterogeneity (e.g., endocrine cell-expressed miR-375 in pancreatic ductal adenocarcinoma). Co-registration with cell type-specific protein markers indicated a previously unappreciated contribution of tumor microenvironment elements to altered miRNA expression. Thus, the source cell(s) of altered miRNA should be carefully considered when designing and interpreting miRNA-based diagnostic assays. The results herein also have implications for miRNA-based therapeutic interventions aimed at restoring basal miRNA activity; since the source cell(s) of miRNA deregulation could be the cancer cells, reactive stroma, infiltrating immune cells and/or other involved cell types, different targeted delivery strategies may be required for different miRNAs (Sempere & Kauppinen (2009) supra). These findings underscore the necessity of spatial characterization of miRNA expression to determine whether the cancer cells, supportive and/or reactive microenvironment elements are the principal source of miRNA deregulation in solid tumors. This information is essential to understand the role of miRNAs in cancer initiation and progression as well as to interpret the impact of miRNA changes in a diagnostic test. Accordingly, the instant method finds application in the detection, classification, prevention, diagnosis, prognosis as well as treatment of cancer.

### Example 1

### Materials and Methods

Processing and Procurement of Tissue Specimens.

Tissue specimens were obtained through the research pathology services from the Tissue Bank at Dartmouth-Hitchcock Medical Center, Lebanon, N.H. These surgical specimens were processed in the surgical pathology laboratory using an institutional standardized protocol. Briefly, surgical specimens were sectioned in 2 mm slices and fixed in 10% formalin for up to 24 hours and then were paraffin-embedded in fully-automated Shandon Pathcentre instrument using a standard overnight procedure (2×10% formalin for 80 minutes, ethanol series 75% to 100 in 6 sequential 45-minute steps, 2× xylenes for 45 minutes and 4× paraplast for 45 minutes).

Probe Design and Hybridization Conditions.

LNA-modified DNA probes were designed to have a predicted melting temperature (Tₘ) between about 75° C. to 78° C. (see Table 1) following the general strategy as previously described (Valoczi, et al. (2004) Nucleic Acids Res. 32(22):e175). Briefly, LNA-modified nucleotides were intercalated at every third nucleotide among DNA nucleotides. When this strategy rendered a probe with a Tₘ lower than 70° C., more LNA-modified nucleotides were introduced, which were intercalated at every second or third position. C or G for LNA modifications were preferentially selected. The Tₘ of the probe against complementary miRNA sequence was calculated using LNA design SciTools (Integrated DNA Technologies Inc., Coralville, Iowa), with default parameters, except for [Na+] which was set to 100 mM. When ordering probes from IDT, these are the codes for the hapten addition onto a terminal 5′ and/or 3′ extra T nucleotides (these Ts are not part of the complementary miRNA sequence): /5biosg/t, /55Br-dU/tt, /5-DigN/t, /56-FAM/t, /5Biosg/t[ . . . ]t/3Bio/, /55Br-dU/tt[ . . . ]tt/i5Br-dU/t, /5-DigN/t[ . . . ]t/3-Dig_N/, and /56-FAM/t[ . . . ]t/36-FAM/. When ordering from EXIQON A/S (Vedbaek, Denmark), these haptens can be added onto the 5′ and/or 3′ end of the probe.

TABLE 1

| | | | SEQ |
|---|---|---|---|
| | | | ID |
| RNA | Tₘ | Probe Sequence | NO: |
| let-7a | 78.3 | AA+CTA+TA+CAA+CC+TA+CTA+CCT+CA | 1 |
| miR-21 | 73.8 | T+CAA+CAT+CA+GT+CTG+ATA+AG+CTA | 2 |
| miR-24 | 80 | CT+GTT+CCT+GCT+GAA+CTG+AGC+CA | 3 |
| miR-34a | 80 | A+CAA+CCA+GCT+AAG+ACA+CTG+CCA | 4 |
| miR-34c-5p | 79 | GCA+ATC+AGC+TAA+CTA+CA+CTG+CCT | 5 |
| miR-125b | 77.2 | TC+ACA+AGT+TAG+GGT+CTC+AGG+GA | 6 |
| miR-126 | 73.4 | CG+CAT+TAT+TAC+TCA+CGG+TAC+GA | 7 |
| miR-141 | 75 | C+CAT+CTT+TAC+CA+GA+CA+GTG+TTA | 8 |
| miR-143 | 73.4 | GA+GCT+ACA+GTG+CTT+CAT+CT+CA | 9 |
| miR-145 | 79 | AAG+GGA+TTC+CTG+GGA+AAA+CTG+GAC | 10 |
| miR-155 | 74 | T+TA+AT+GCT+AAT+CGT+GAT+AG+GG+GT | 11 |
| miR-196a | 76.8 | CC+CAA+CAA+CAT+GA+AA+CT+AC+CTA | 12 |
| miR-200b | 73.7 | GT+CAT+CAT+TA+CCA+GG+CA+GTA+TTA | 13 |
| miR-205 | 75.6 | CA+GAC+TCC+GGT+GGA+ATG+AAG+GA | 14 |
| miR-214 | 80.4 | CT+GCC+TGT+CTG+TGC+CTG+CTGT | 15 |
| miR-221 | 80.8 | GA+AA+CC+CAG+CAGA+CAA+TGT+AG+CT | 16 |
| miR-375 | 75 | TC+ACG+CGA+GCC+GAA+CGA+ACA+AA | 17 |
| miR-451 | 76.7 | AAA+CT+CAG+TA+AT+GG+TAA+CG+GT+TT | 18 |
| miR-10b | 76.4 | CA+CAA+ATT+CGG+TT+CTA+CA+GG+GTA | 19 |
| let-7b | 77.5 | AA+CCA+CA+CAA+CCT+ACT+ACC+TCA | 20 |
| let-7f | 74.7 | AA+CTA+TA+CA+AT+CTA+CTA+CCT+CA | 21 |
| let-7g | 78.4 | AA+CTG+TA+CA+AA+CTA+CTA+CCT+CA | 22 |
| miR-7 | 73.6 | A+CAA+CAA+AAT+CAC+TAG+TCT+TC+CA | 23 |
| miR-15b | 72.8 | T+GTA+AAC+CAT+GAT+GTG+CTG+CTA | 24 |
| miR-17 | 73.4 | C+TAC+CTG+CAC+TGT+AAG+CAC+TT+TG | 25 |
| miR-18a | 77.3 | C+TAT+CTG+CA+CTA+GAT+GCA+CCT+TA | 26 |
| miR-27b | 76.1 | G+CA+GAA+CTT+AGC+CAC+TGT+GAA | 27 |
| miR-29b | 75.8 | AA+CA+CT+GAT+TT+CAA+ATG+GTG+CTA | 28 |
| miR-30a | 73.7 | CTT+CCA+GTC+GAG+GAT+GTT+TA+CA | 29 |
| miR-31 | 78.2 | AG+CTA+TGC+CAG+CAT+CTT+GCCT | 30 |
| miR-92 | 79.3 | A+CAG+GCC+GGG+ACA+AGT+GCA+ATA | 31 |
| miR-100 | 77.4 | CA+CAA+GTT+CGG+AT+CTA+CGG+GTT | 32 |
| miR-106b | 73.8 | AT+CTG+CA+CT+GTC+AGC+ACT+TTA | 33 |
| miR-107 | 75.8 | TG+ATA+GCC+CTG+TAC+AAT+GCT+GCT | 34 |
| miR-122 | 79.8 | C+AAA+CAC+CAT+TGT+CA+CA+CTC+CA | 35 |
| miR-124 | 79 | GG+CAT+TCA+CCG+CGT+GCC+TTA | 36 |
| miR-142-5p | 71.3 | AG+TA+GTG+CT+TT+CTA+CTT+TA+TG | 37 |
| miR-146 | 75.5 | AA+CCC+AT+GG+AA+TTC+AGT+TCT+CA | 38 |
| miR-148a | 73.7 | AC+AAA+GTT+CT+GTA+GT+GCA+CT+GA | 39 |
| miR-181a | 75.9 | A+CTC+ACC+GAC+AGC+GTT+GAA+TGTT | 40 |
| miR-181b | 78.8 | A+CCC+ACC+GAC+AGC+AAT+GAA+TG+TT | 41 |
| miR-191 | 76.9 | CA+GCT+GCT+TTT+GGG+ATT+CC+GT+TG | 42 |
| miR-196b | 75.4 | CC+CAA+CAA+CA+GG+AA+ACT+ACC+TA | 43 |
| miR-200c | 75 | TC+CAT+CAT+TAC+CCG+GCA+GTA+TTA | 44 |
| miR-203 | 70.4 | C+TA+GTG+GT+CCT+AAA+CAT+TT+CAC | 45 |
| miR-204 | 75.3 | AG+GCA+TAG+GAT+GAC+AAA+GG+GAA | 46 |
| miR-206 | 77.7 | CC+ACA+CAC+TTC+CT+TA+CA+TTC+CA | 47 |
| miR-210 | 77.6 | TCA+GCC+GCT+GTC+ACA+CGC+ACAG | 48 |
| miR-216 | 79 | T+CA+CA+GTT+GC+CAG+CTG+AGA+TTA | 49 |
| miR-217 | 74.2 | TC+CAA+TCA+GTT+CCT+GAT+GCA+GTA | 50 |
| miR-222 | 77.1 | AC+CCA+GTA+GC+CA+GAT+GTA+GCT | 51 |
| miR-335 | 69.5 | A+CAT+TT+TTC+GTT+ATT+GCT+CTT+GA | 52 |
| miR-373 | 77 | A+CAC+CCC+AAA+ATC+GAA+GCA+CTTC | 53 |
| miR-376 | 72.5 | AC+GT+GG+AT+TTT+CCT+CTA+TG+AT | 54 |
| miR-520c | 75.2 | CA+GAA+AGT+GCT+TC+CCT+CTA+GAG | 55 |
| miR-136 | 72 | TC+CAT+CAT+CAA+AA+CAA+ATG+GAGT | 56 |
| U6 snRNA | 75 | CGTGTCATCCTTGCGCAGGGGCCATGCTAA | 57 |
| | | TCTTCTCTGT | |
| 18S rRNA | 79.3 | GGGCAGACGTTCGAATGGGTCGTCGCCGCC | 58 |
| | | ACGGG | |

“+N” denotes LNA-modified nucleotide.

Combined ISH/IHC Assay.

Four μm tissue sections were mounted on positively-charged barrier frame slides, de-waxed in xylenes, and re-hydrated through an ethanol dilution series (100% to 25%). Tissue sections were digested with 5 μg/mL of proteinase K for 20 minutes at 37° C. to facilitate probe penetration and exposure of miRNA species. To minimize non-specific binding based on charge interactions, tissue was subject to a brief acetylation reaction (66 mM HCl, 0.66% acetic anhydride (v/v) and 1.5% triethanolamine (v/v) in RNase-free water). Then, tissue sections were pre-hybridized at the hybridization temperature (see Table 2) for 30 minutes in pre-hybridization solution, which was composed of 50% deionized formamide, 5× Sodium chloride-Sodium citrate buffer (SSC), 1×Denhardt's solution, 500 μg/ml yeast tRNA, 0.01% TWEEN. Pre-hybridization solution was replaced with 200 μL of hybridization solution containing 10 pmol of the hapten-labeled LNA probe and tissue were incubated for 90 minutes at T_{hyb} and washed three times for 10 min in SSC buffer at the established stringency of SSC (see Table 2 for details).

TABLE 2

| RNA | ° C.^{a} | SSC^{b} | Expression^{c} |
|---|---|---|---|
| let-7a | 50 | 0.5X | Various |
| miR-21 | 45 | 0.5X | TAFs, cancer cells |
| miR-24 | 37 | 0.5X | Various |
| miR-34a | 50 | 0.1X | Epithelia, others |
| miR-34c-5p | 37 | 0.5X | Epithelia |
| miR-125b | 45 | 0.5X | Fibroblasts, epithelia |
| miR-126 | 45 | 0.5X | Endothelia |
| miR-141 | 37 | 0.5X | Epithelia |
| miR-143 | 50 | 0.5X | Various |
| miR-145 | 50 | 0.1X | Smooth Muscle Cells |
| miR-155 | 50 | 0.5X | Leukocytes |
| miR-196 | 37 | 0.5X | Cancer cells |
| miR-200b | 37 | 0.5X | Various |
| miR-205 | 50 | 0.1X | Myo/basal epithelia |
| miR-214 | 50 | 0.1X | Various |
| miR-221 | 37 | 0.5X | Various |
| miR-375 | 45 | 0.5X | Endocrine cells |
| miR-451 | 45 | 0.5X | Erythrocytes |
| U6 snRNA | 50 | 0.5X | Ubiquitous |
| 18S rRNA | 50 | 0.5X | Ubiquitous |

^{a}Incubation temperature of hybridization and washing steps.
^{b}Concentration of SSC in washing steps.
^{c}Summary of predominant cell type(s) of miRNA expression in normal and tumor tissues from breast, colon, lung, pancreas and/or prostate.
“Various” indicates a complex pattern of expression as regards to the cell type(s) in which the miRNA is expressed and/or the direction of miRNA expression changes within cancer cells.

At this point, tissue slides were loaded onto the BIOGENEX 16000 staining machine (BIOGENEX Laboratories Inc, San Ramon, Calif.) which was programmed to dispense 400 μL of appropriate reagent per step. Slides were treated with 3% H₂O₂ to inactivate endogenous peroxidase and block with 5% bovine serum albumin (BSA) in PBS (w/v). Followed by primary and secondary antibody incubation in PBT (1% BSA (w/v), 0.1% TWEEN 20 (v/v) in PBS) and washes in PBST (0.01% TWEEN 20 (v/v) in PBS), tyramine-conjugated fluorochrome was applied to the slide and the TSA reaction was allowed to proceed for 10-30 minutes. Sequential TSA rounds for detection of other miRNAs, non-coding RNAs or proteins followed the same protocol. Finally, slides were profusely washed with PBST and mounted with anti-fading PROLONG gold solution (Invitrogen, Carlsbad, Calif.) with or without DAPI (for nuclear counterstaining).

Antibodies used in IHC analysis are listed in Table 3, whereas the antibodies used in hapten detection are listed in Table 4. Secondary antibodies are listed in Table 5.

TABLE 3

| Epitope | Dilution | Retrieval | Clone Name | Company |
|---|---|---|---|---|
| Amylase | 1:200^{a} | AR1 | G-10 | Santa Cruz |
| CK5/6 | 1:50^{a} | AR1+ | | Biocare |
| CK7 | 1:100^{a} | 0 | OV-TL12/30 | Biogenex |
| CK8/18 | 1:50^{a} | 0 | 5D3 | Biogenex |
| CK14 | 1:100^{a} | AR2 | | Biocare |
| CK19 | 1:200^{a} | 0 | RCK108 | Biogenex |
| CK20 | 1:200^{a} | 0 | Ks20.8 | Dako |
| CD31 | 1:100^{a} | AR1 | JC70A | Dako |
| CD45 | 1:100^{a} | AR1 | 2D1 | BD Pharmingen |
| E-cadherin | 1:200^{b} | AR1 | 24E10 | Cell Signaling |
| ER | 1:10^{a} | AR1+ | 1D5 | Biogenex |
| Glucagon | 1:500^{a} | AR1 | K79bB10 | Abcam |
| HER2 | 1:20^{a} | AR1+ | CB11 | Biogenex |
| Insulin | 1:500^{c} | 0 | | Dako |
| Ki-67 | 1:200^{a} | AR1 | MM1 | Novocastra |
| pAKT | 1:50^{b} | 0 | 736E11 | Cell Signaling |
| P53 | 1:200^{a} | AR1 | DO-1 | Santa Cruz |
| PCNA | 1:300^{a} | AR1+ | PC10 | Novocastra |
| PR | 1:25^{a} | AR1+ | PR88 | Biogenex |
| PTEN | 1:100^{b} | AR1 | 138G6 | Cell Signaling |
| Smooth | 1:500^{a} | 0 | 1A4 | Biogenex |
| Muscle Actin | | | | |
| Somatostatin | 1:100^{a} | 0 | | Zymed |
| Tubulin | 1:1000^{d} | 0 | YL1/2 | Abcam |
| Vimentin | 1:1000^{a} | AR1 | V9 | Biogenex |

^{a}Mouse,
^{b}Rabbit,
^{c}Guinea pig, and
^{d}Rat.
Protein detection was compatible with previous ISH procedures without further treatment (0) or followed by heat-induced epitope retrieval by a 20-minute incubation in 95° C. water bath with pre-warmed citrate-based antigen unmasking solution (AR1; Vector Laboratories) or Reveal (AR2; Biocare Medical), or by heating in decloaking chamber (Biocare Medical) with default program (1 minute at 121° C.) with citrate-based antigen unmasking solution (AR1+).

TABLE 4

| Epitope | Dilution* | Name (clone) | Company |
|---|---|---|---|
| Biotin | 1:7000 | Streptavidin/HRP | Invitrogen |
| BrdU | 1:1000^{a} | Anti-BrdU | Novus Biologicals |
| DIG | 1:1000^{b} | Anti-DIG/HRP | Roche |
| FITC, FAM | 1:200^{c} | Anti-FITC/HRP | Dako |

^{a}Rat,
^{b}Sheep, and
^{c}Rabbit.
*Protein detection was compatible with previous ISH procedures without further treatment.

TABLE 5

| Epitope | Dilution | Name (clone) | Company |
|---|---|---|---|
| Guinea pig | 1:500^{a} | Anti-guinea pig/CY3 | Jackson |
| Fc | | | ImmunoResearch |
| Goat Fc | 1:500^{b} | Anti-goat/HRP | BioRad |
| Mouse Fc | 1:500^{a} | Anti-mouse/HRP | BioRad |
| Rat Fc | 1:1000^{a} | Anti-rat/HRP | Calbiochem |
| Rabbit Fc | 1:500^{a} | Anti-rabbit/HRP | BioRad |
| Sheep Fc | 1:500^{b} | Anti-sheep/HRP | Calbiochem |

^{a}Goat and
^{b}Rabbit.
*Protein detection was compatible with previous ISH procedures without further treatment.

Preparations of tyramide-conjugated fluorescent substrates for miRNA and protein detection are listed respectively in Table 6 and Table 7. Preparation of the reagents was based on the Davidson protocol for FITC tyramide substrate. See also Vize, et al. (2009) Nat. Protoc. 4 (6):975-83 for additional information.

TABLE 6

| Substrate | Dilution (miRNA) | Incubation time (miRNA) |
|---|---|---|
| ALEXA fluor 647 | Not determined | Not determined |
| AMCA | 1:300-1:500 | 15 min. (10-30 min.) |
| DYLIGHT405 | 1:50-1:100 | 15 min. (10-30 min.) |
| DYLIGHT594 | 1:50-1:100 | 15 min. (10-30 min.) |
| DYLIGHT649 | Not determined | Not determined |
| DYLIGHT680 | Not determined | Not determined |
| FITC | 1:100-1:200 | 15 min. (10-30 min.) |
| Rhodamine | 1:500 | 15 min. (10-30 min.) |

TABLE 7

| | Dilution | Incubation time |
|---|---|---|
| Substrate | (protein) | (protein) |
| ALEXA fluor 647 | 1:100-1:200 | 15 min. (10-30 min.) |
| AMCA | 1:500-1:1000 | 10 min. (5-20 min.) |
| DYLIGHT405 | 1:100-1:200 | 15 min. (10-30 min.) |
| DYLIGHT594 | 1:100-1:200 | 15 min. (10-30 min.) |
| DYLIGHT649 | 1:100-1:200 | 15 min. (10-30 min.) |
| DYLIGHT680 | 1:100-1:200 | 15 min. (10-30 min.) |
| FITC | 1:200-1:400 | 10 min. (5-20 min.) |
| Rhodamine | 1:1000 | 10 min. (5-20 min.) |

ALEXA fluor 647-NHS ester was obtained from Invitrogen; AMCA-NHS ester, DYLIGHT405-NHS ester, DYLIGHT594-NHS ester, DYLIGHT649-NHS ester, DYLIGHT680-NHS ester, Fluorescein-NHS ester, and Rhodamine-NHS ester were obtained from Pierce; and Tyramine hydrochloride, dimethyl formamide (DMF), and triethylamine (TEA) were obtained from Sigma. For acetylation reactions, triethanolamine was used.

Coupling Reaction.

Work was conducted under a chemical hood in a dry environment. If stock reagents were frozen, tubes were equilibrated at room temperature and wiped with a paper towel to remove any moisture. Fresh reagents only were used. The coupling reaction including dissolving the fluorochrome-NHS ester at 10 mg/mL stock in DMF and preparing a DMF-TEA solution (100:1 v/v). The tyramine was dissolved at 10 mg/mL in DMF-TEA and the needed volume of tyramide/DMF-TEA was added to the fluorochrome-NHS ester/DMF to achieve a molar ratio of 1:1. The mixture was incubated in the dark at room temperature for 2 hours. One volume of 100% ethanol was added and the reagent was stored at −20° C.

Microscopy and Image Analysis.

Fluorescent images were captured with an F-view II monochrome camera (OLYMPUS) mounted on an OLYMPUS BX60 microscope with filter cubes for AMCA/DYLIGHT405/DAPI, Fluorescein, Rhodamine/CY3, DYLIGHT594/TR, ALEXA647/DYLIGHT649/CY5, and DYLIGHT680/CY5.5 using CELLSENS software package (Olympus, Center Valley, Pa.). Grayscale TIF files were converted to RGB TIF files for image analysis with intensity measurement tools of Image-Pro Plus software package (Media Cybernetics, Bethesda, Md.).

### Example 2

### Optimization miRNA Detection in FFPE Specimens

Conditions for ISH were systematically tested and optimized. It was determined that digestion with Proteinase K at 5 μg/mL for 20 minutes at 37° C. provided optimal results for archived FFPE human breast, colon, lung, pancreas and prostate tissues. To further improve the sensitivity of detection, probes were designed with a hapten at both the 5′ and 3′ ends on extra T nucleotides that were not part of the miRNA complementary sequence. In addition a secondary antibody step (antibody sandwich amplification) was introduced using HRP-conjugated antibody against the primary anti-hapten antibody. By using double hapten-tagged (5′ and 3′ ends; hapten2X) vs. single hapten-tagged (5′ end; hapten1X) probes, a greater than 3-fold increase in sensitivity of signal detection of miRNAs and other non-coding RNA species was observed. Importantly, this improved protocol allowed for the visualization of the expression of miRNAs that had previously fallen below detection limit of ISH assays.

### Example 3

### Experimental Reproducibility of Continuous Measurements

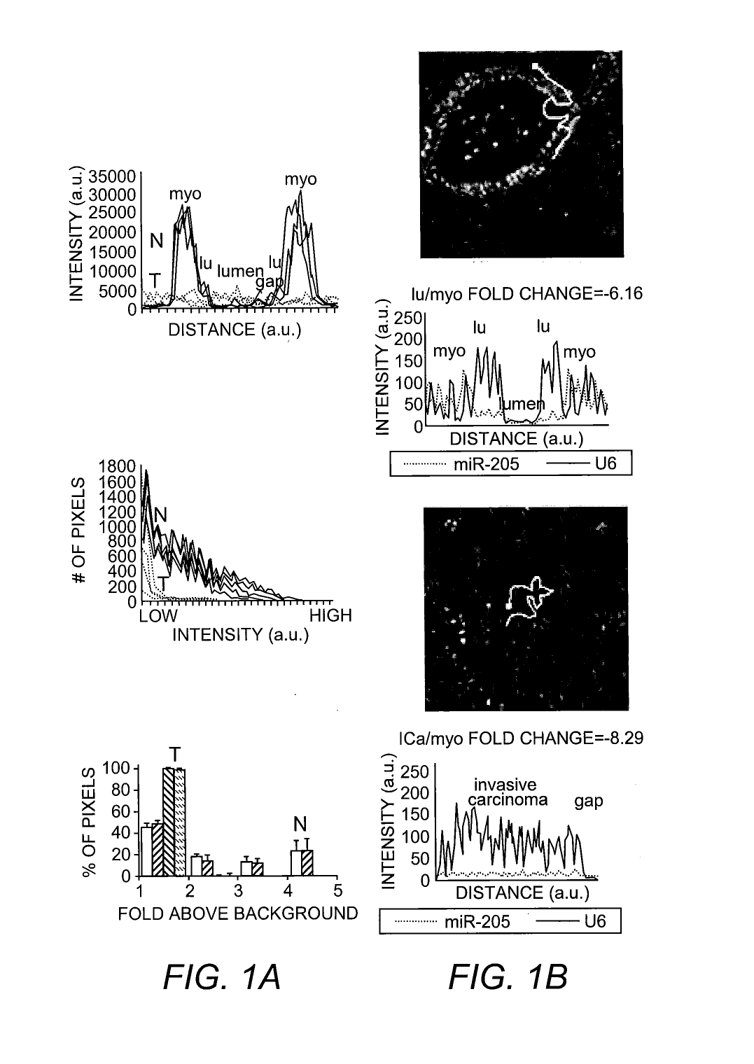

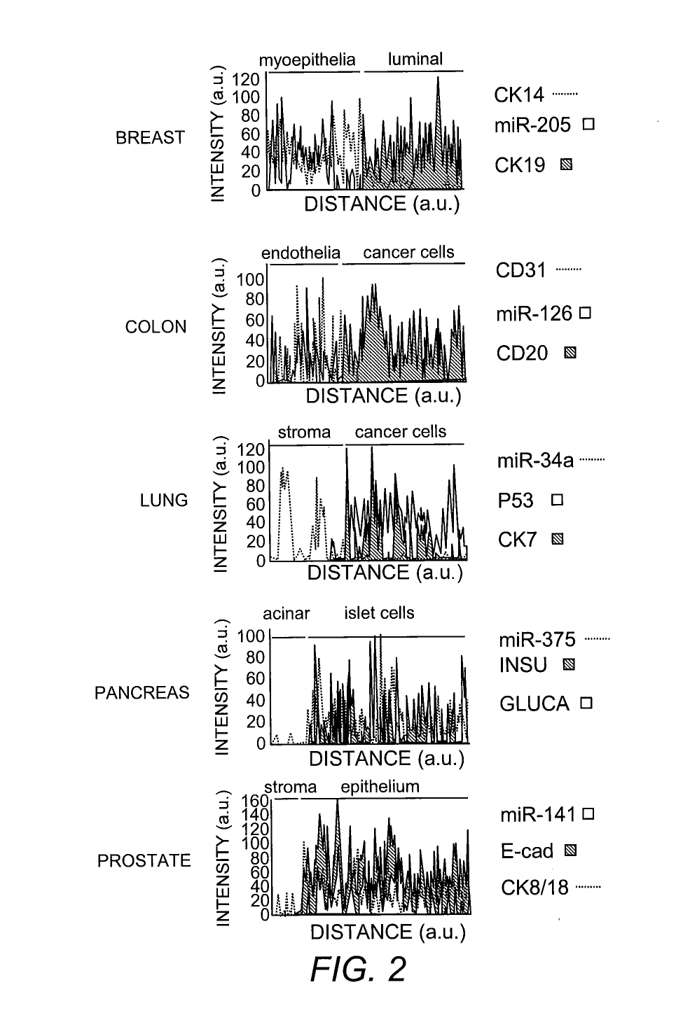

Given the importance of reproducibility in the clinical setting, the intra- and inter-experimental variation of the ISH method was assessed. Consecutive sections of matched normal and tumor breast tissue were mounted on the same slide and groups of three of these slides were independently subject to ISH method on separate days for detection of miR-205. To minimize experimental variation due to operator, experiments were performed in an FDA-approved automated staining station (BIOGENEX i6000 machine) for all steps after probe washes. Signal intensity and overall staining pattern of miR-205 expression was highly concordant as determined by visual examination and by computer-assisted image analysis. Heat map plots and line profile analysis tools were used to measure intensity of miR-205 signal as an effort to objectivize, standardize and eventually automate a scoring system for miRNA expression (FIGS. 1A and 1B). Similar analytical tools were used for signal quantification and co-localization studies of miRNA and protein expression shown in FIGS. 2-4.

### Example 4

### Codetection of miRNAs and Other Non-coding RNAs in FFPE Specimens

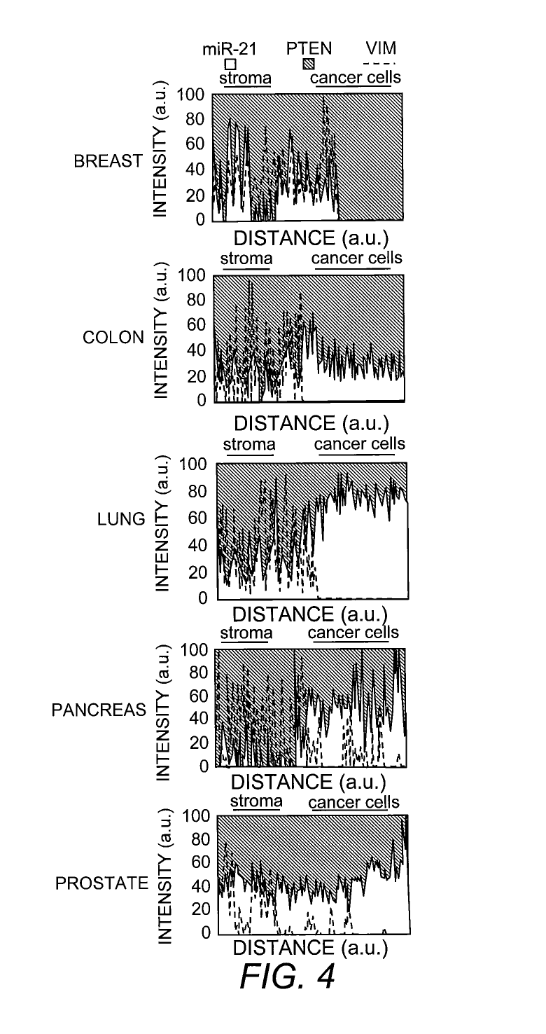

To assess quality and integrity of RNA preservation in the tissue sample and to normalize changes in miRNA expression, several distinct miRNAs and abundant control non-coding RNAs, either small nuclear (sn)RNA U6 or 18S ribosomal (r)RNA were codetected (FIG. 1, FIG. 2, and FIG. 4). Staining with ubiquitously-expressed snRNA U6 and 18S rRNA was also used to identify tissue dehydration (dry spots), spurious edge effects, or other technical problems caused by suboptimal sample handling. Moreover, the codetection of multiple miRNA species was carried out to maximize the amount of information obtained per tissue section, which would be especially important if: i) Tissue availability were limited such as tissue cores of a tissue microarray, core needle biopsies, and cell preparations obtained by fine needle aspiration; and/or ii) Multivariate analysis of miRNAs with opposite expression changes between normal and cancer cells or within different cell types within the tumor lesion yielded a more powerful clinical indicator than either miRNA alone. For codetection of miRNAs and/or control RNAs, probes were utilized that were 5′ and 3′ terminally tagged (hapten2X) with biotin (Bio)-, digoxigenin (DIG)-, 5-bromo-2-deoxyuridine (BrdU)- or 5- (and -6) Carboxyfluorescein (FAM). The desired FAM2X, Bio2X, BrdU2X and/or DIG2X probes were mixed together and annealed with their respective complementary RNA sequences in a single hybridization incubation step. After washes to eliminate unbound probes, codetection of multiple RNA species was achieved by sequential tyramide signal amplification (TSA; Speel, et al. (2006) Methods Mol. Biol. 326:33-60) reactions using different tyramine-conjugated fluorescent substrates. Following this strategy up to five independent RNA and/or protein markers could be codetected on the same tissue section (FIG. 4). AMCA-NHS ester (appearing blue), Fluorescein-NHS ester (appearing green), Rhodamine-NHS ester (appearing red) provided a convenient and economic fluorochrome choice for a three-color co-staining scheme using conventional fluorescence microscopy. A series of DYLIGHT-NHS (Pierce) or ALEXA FLUOR-NHS (Invitrogen) fluorescent compounds with distinct excitation/emission spectra allowed for additional colors. MiRNA detection with commercially available TSA kits was less sensitive than with the reagents and protocol described herein. This may have been due to considerable differences in concentration between the fluorescent substrates in ALEXA FLUOR TSA kit (Invitrogen) and TSA Plus System (PerkinElmer) and the synthesized fluorochromes.

### Example 5

### Codetection of miRNAs and Protein Markers

To ascribe miRNA expression to a specific cell type and to better understand the physiological or pathological status of the cell, codetect of miRNAs and clinically important protein markers was carried out on the same tissue section. Numerous protein markers expressed in normal tissues also display specific expression patterns in tumor tissues that can be especially informative: i) Cytokeratin 19 (CK19) is an epithelial-specific markers and as such widely to identify carcinoma cells; ii) CK5/6 and CK14 are expressed in normal myoepithelial cells and specifically identify the aggressive basal breast cancer subtype (Moriya, et al. (2006) Med. Mol. Morphol. 39(1):8-13); iii) CK7/CK20 signal can be used to discern the organ of origin of certain cancers, for example most primary and metastatic colorectal carcinoma cells are CK7−/CK20+ and lung carcinoma cells are CK7+/CK20− cells (Tot (2002) Eur. J. Cancer 38(6):758-63); iv) Glucagon and insulin are specifically expressed in α and β endocrine pancreatic cells, respectively, and as such are useful markers for establishing the type of endocrine pancreatic tumors (Tomita (2002) Pathol. Int. 52(7):425-32). In addition, miRNAs have been shown to mediate the repression of and/or to be regulated by important oncogenic and tumor suppressor genes (Ventura & Jacks (2009) supra; Sempere & Kauppinen (2009) supra). Thus, codetection of miRNA and protein markers have important clinical applications.

Detection of protein markers was generally compatible with proteinase K digestion and other chemical treatments used in the preceding ISH steps. Some useful epithelial-specific markers such as CK19 and other cytokeratins (e.g., CK7, CK8/18 and CK20) were detected without further tissue processing, while other protein markers required heat-induced epitope retrieval (HIER) to be efficiently detected. Although HIER did not quench fluorescent signal from previous steps, tissue damage and detachment was observed in some slides and signal was diminished by physically washing off the anchored fluorochrome molecules. Thus, the mildest proteinase K treatment (limit incubation time) and HIER treatment (limit the temperature of HIER and/or incubation time) should be used for optimal codetection conditions of miRNAs and proteins. To further demonstrate use of the instant method, several examples of miRNAs frequently associated with cancer were detected to demonstrate the feasibility of codetection of miRNA and protein markers and to illustrate potential uses of miRNAs as novel biomarkers. Table 1 also lists a general summary of the expression pattern of the 18 miRNAs that were analyzed in this study.

### Example 6

### Co-Localization of miRNAs and Cell-Type Specific Protein Markers

It has been previously reported that expression of miR-205 is confined to a subpopulation of mammary epithelial cells and that there is a positive trend of association between miR-205 expression and a favorable clinical outcome in patients of the basal breast cancer subtype (ER-PR-HER2-) (Sempere, et al. (2007) supra). Co-staining with CK14 (myoepithelial cells) and CK19 (luminal epithelial cells) protein markers confirmed myoepithelia-restricted expression of miR-205 in breast tissue (FIG. 2). Recent reports based on RT-PCR analyses indicated that miR-205 expression could be useful to distinguish squamous and adenocarcinoma subtypes of lung cancer (Bishop, et al. (2010) Clin. Cancer Res. 16(2):610-9; Lebanony, et al. (2009) J. Clin. Oncol. 27(12):2030-7). In an independent study, low levels of miR-205 were associated with poor prognosis in head and neck squamous carcinomas (Wu, et al. (2009) Cell Res. 19(4):439-48). Thus, a combined ISH/IHC assay for detection of miR-205 and basal/squamous epithelial cell marker (e.g., CK14) would increase the interpretative power of miR-205 expression changes and could be further implemented as a diagnostic and/or prognostic tool in solid tumors.

Expression profiling analyses detected miR-126 at lower levels in colorectal and other carcinomas compared to normal tissue (Tavazoie, et al. (2008) Nature 451(7175):147-52; Guo, et al. (2008) Genes Chromosomes Cancer 47(11):939-46; Diaz, et al. (2008) Genes Chromosomes Cancer 47(9):794-802; Xiao, et al. (2009) Gut 59:579-585; Miko, et al. (2009) Exp. Lung Res. 35(8):646-64). Co-staining with CD31/PECAM-1 (endothelial cells) and CK20 (epithelial cells) protein markers indicated an endothelial-specific expression of miR-126 in colonic and other tissues (FIG. 2). ISH results herein indicate that miR-126 expression changes in tumor tissue could mainly reflect differences in distribution of blood vessels compared to normal tissue, and perhaps, differences in the integrity and/or structure of the neovasculature. Similarly, expression profiling analyses detected miR-375 at lower levels in pancreatic tumor tissues compared to normal pancreas (Mardin & Mees (2009) Ann. Surg. Oncol. 16(11):3183-9). Co-staining with insulin and glucagon confirmed endocrine-restricted expression of miR-375 (FIG. 2) in pancreatic tissue. ISH results herein indicate that miR-375 is expressed in cell types that do not contribute to pancreatic ductal adenocarcinoma formation.

Moreover, miR-141 was reported as a potential serum biomarker for prostate cancer detection (Mitchell, et al. (2008) Proc. Natl. Acad. Sci. USA 105(30):10513-8). Independent studies have shown that members of the miR-141/miR-200 family act to maintain epithelial genetic programs via binding to 3′UTR sites on the mRNAs of ZEB1/2 zing finger transcriptional repressors (Burk, et al. (2008) EMBO Rep. 9(6):582-9; Gregory, et al. (2008) Nat. Cell Biol. 10(5):593-601; Korpal, et al. (2008) J. Biol. Chem. 283(22):14910-4; Park, et al. (2008) Genes Dev. 22(7):894-907; Hurteau, et al. (2007) Cancer Res. 67(17):7972-6). Co-staining with CK8/18 and E-cadherin indicated that expression of miR-141 was confined to prostatic epithelium (FIG. 2). ISH results herein indicated that the presence of miR-141 in the peripheral circulation of these patients may closely reflect changes within the neoplastic cells.

As a further example, codetection of miR-21, p53 and E-cadherin are of use in the diagnosis of colon cancer.

### Example 7

### Value of miRNAs as Functional Biomarkers

MiR-34 family members (miR-34a,b,c) are transcriptionally activated by the p53 tumor suppressor gene in response to DNA damage and mitogenic signals (He, et al. (2007) Nat. Rev. Cancer 7(11):819-22). Consistent with a tumor suppressive role, miR-34s were detected at lower levels in breast, lung and other solid tumors by expression profiling analyses in whole tissue biopsies (Barbarotto, et al. (2008) supra). Co-staining with CK7 (epithelial marker), p53 (tumor suppressor gene) and Ki-67 (proliferation marker) protein markers indicated altered expression of miR-34a within malignant cells in lung (FIG. 2) and breast cancer. Detection of p53 expression by IHC is suggestive of discrete or point mutations that interfere with p53 tumor suppressive functions, negative IHC staining is not informative because it cannot distinguish other mutated forms or chromosomal deletion of p53 from wild-type and functional p53 gene (Zhu, et al. (2006) J. Clin. Pathol. 59(8):790-800). A miR-34-based ISH assay can inform by proxy of p53 activity status and provide a better prognostic indicator than a p53-based IHC assay alone.

### Example 8

### miRNAs and the Immune Response

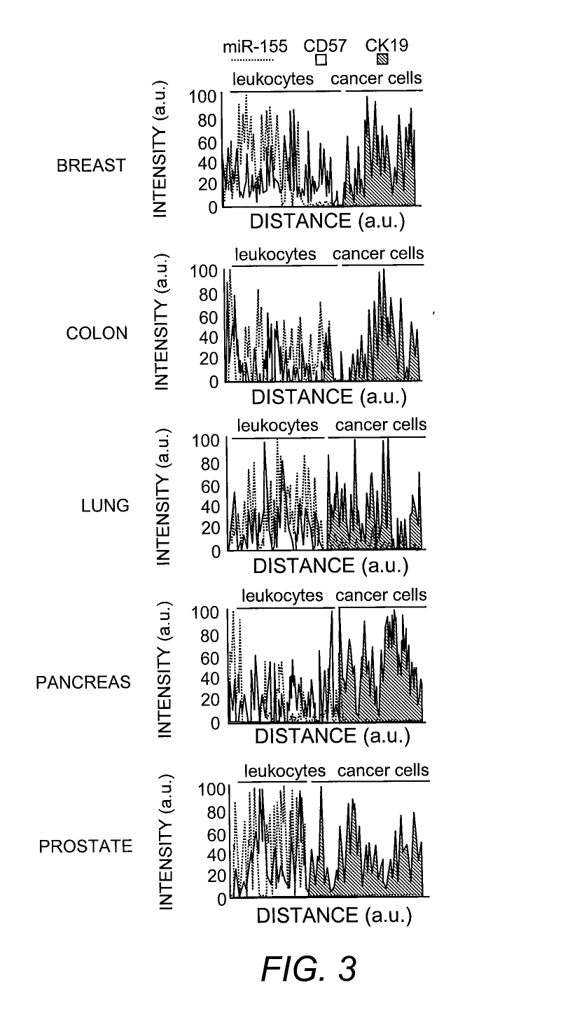

High levels of miR-155 expression have been frequently detected in leukemias, lymphomas as well as breast, lung, pancreas and thyroid carcinomas (Tili, et al. (2009) Int. Rev. Immunol. 28(5):264-84; Faraoni, et al. (2009) Biochim. Biophys. Acta 1792(6):497-505). High levels of miR-155 correlated with poor outcome in patients with lung and pancreatic cancer (Yanaihara, et al. (2006) Cancer Cell 9(3):189-98; Greither, et al. (2010) Int. J. Cancer 126(1):73-80). In vitro and in vivo studies have unraveled an oncogenic role for miR-155 in hematologic and solid tumors (Tili, et al. (2009) supra; Faraoni, et al. (2009) supra; Jiang, et al. (2010) Cancer Res. 70(8):3119-27; Valeri, et al. (2010) Proc. Natl. Acad. Sci. USA 107(15):6982-7). A large body of evidence also attributes an important role for miR-155 in the immune system as a mediator of lymphoid and myeloid cell responses to infection and inflammation (Tili, et al. (2009) supra; Faraoni, et al. (2009) supra; Okada, et al. (2010) Int. J. Biochem. Cell Biol. February 6; Tsitsiou & Lindsay M A (2009) Curr. Opin. Pharmacol. 9(4):514-20), which is further supported by immunological deficiency exhibited by mir-155 knockout mouse models (That, et al. (2007) Science 316(5824):604-8; Rodriguez, et al. (2007) Science 316(5824):608-11). Co-staining with CK19 and CD45 (leukocyte marker) indicated that expression of miR-155 was predominantly confined to a subpopulation of infiltrating immune cells in breast, colorectal, lung, pancreas and prostate tumor lesions (FIG. 3). Further characterization with specific lymphoid and myeloid markers will establish the identity of miR-155 expressing cells and whether a common cell population is present in solid tumors or distinct subpopulations in each tumor type. Nonetheless, these results indicate that the majority of miR-155 signal detected by RT-PCR assays in whole tissue biopsies or blood samples likely emanates from tumor-associated immune cells, and thus this fact should be taken into consideration when interpreting RT-PCR results. In some tumor specimens, a low level of miR-155 expression was detected within cancer cells (FIG. 3). Since ISH is not as sensitive as other detection methods, particularly real time RT-PCR, undetectable or low levels of expression of miR-155 by ISH may still be functionally important within cancer cells. Recent studies reported detection of miR-155 by ISH within immune cells and cancer cells in colorectal adenocarcinoma lesions and predominantly within cancer cells in pancreatic adenocarcinoma lesions (Valeri, et al. (2010) supra; Ryu, et al. (2010) Pancreatology 10(1):66-73).

### Example 9

### Correlation of miRNA and Target Gene Expression in Clinical Samples

Like miR-155, high levels of miR-21 expression have been frequently detected in hematologic and solid tumors and miR-21 is considered to be an important oncogenic miRNA based on functional studies in cancer cell lines and animals models (Sempere & Kauppinen (2009) supra; Krichevsky & Gabriely (2009) J. Cell. Mol. Med. 13(1):39-53). However, it was found that in solid tumors the source cells of altered expression for miR-21 and miR-155 were strikingly different. Higher levels of miR-21 expression were detected within cancer cells and tumor-associated fibroblasts (TAFs) compared to matched normal tissues. Generally, miR-21 was predominantly expressed at higher levels within cancer cells in lung, pancreas and prostate cancer; whereas in breast and colorectal cancer, higher levels of miR-21 expression were more apparent within tumor associated fibroblasts (TAF) as supported by co-staining with the mesenchymal markers vimentin and smooth muscle actin (FIG. 4). These observed patterns of miR-21 accumulation were consistent with previous studies in breast and lung cancer and an independent study in colorectal adenocarcinoma (Sempere, et al. (2007) supra; Yamamichi, et al. (2009) supra; Liu, et al. (2010) supra), but were in disagreement with reported nuclear-enriched staining of miR-21 within cancer cells in pancreatic adenocarcinoma lesions (Dillhoff, et al. (2008) supra).

In vitro studies indicated that that miR-21 inhibits the expression of tumor suppressive PTEN in colon, liver and pancreatic cancer cell lines (Krichevsky & Gabriely (2009) supra; Park, et al. (2009) Pancreas 38(7):e190-e199). To test this interaction in a clinical setting, the co-expression of miR-21 and PTEN protein levels were analyzed in carcinoma lesions of the breast, colon, lung, pancreas and prostate tissue. In this sample size, PTEN expression exhibited a partially inverse pattern to that of miR-21 both within cancer cells and the stromal compartment, providing some support for this regulatory interaction, especially in lung adenocarcinomas (FIG. 4). This example also underscores the feasibility of codetecting a miRNA and protein levels of target gene(s) on the same tissue section. Maspin, PDCD-4, RECK, TIMP3 and TPM1 are among other miR-21 target genes (Sempere & Kauppinen (2009) supra; Krichevsky & Gabriely (2009) supra) that could be studied using this combined ISH/IHC assay and whose regulatory interactions may be clinically informative.

Moreover, miR-146a expression has been correlated with cervical cancer.

### Example 10

### Characterization of miRNA-10b Expression in Pancreatic Ductal Adenocarcinoma and its Precursor Lesions

Pancreatic ductal adenocarcinoma (PDAC) is the fourth-leading cause of cancer related mortality in the US with a median survival rate of 6 months. PDAC's lethal nature stems from its unique biology: aggressive local invasion, marked desmoplasia, resistance to apoptosis and chemotherapy, and metastatic potential. Non-coding microRNAs are involved in initiation and dissemination of many types of cancer, including PDAC. MiR-10b, induced by the transcription factor Twist, has been found to target the expression of HOXD10, promoting epithelial to mesenchymal transformation (EMT). Accordingly, miR-10b expression was analyzed in PDAC by comparing the spatial expression of miR-10b in PDAC with the normal pancreas, intraductal papillary mucinous neoplasms (IPMNs), benign cysts, and neuroendocrine tumors.

Methods.

In situ detection of microRNAs was performed using Locked Nucleic Acid (LNA) bicyclical high-affinity RNA analogs, followed by codetection of specific proteins by immunohistochemistry on the same formalin-fixed, paraffin-embedded tissue sections. Fluorescent images of miR-10b, snRNA U6, Cytokeratin 19 (CK19), HOXD10 and Twist were captured with a monochrome camera, and analyzed for miR-10b expression in CK19-positive cells (CK19+) using the optical intensity analytical tools of IMAGEPRO Plus software.

Results.

The expression of miR-10b was initially characterized in 10 resected PDAC samples. The expression of miR-10b was significantly higher in the CK19+ cancer cells compared to normal ductal epithelial cells (4173±1468 vs. 1078±238 optical density units; p<0.004), and was associated with high levels of Twist and low levels of HOXD10. Next, miR-10b expression was analyzed in 106 fine needle aspirates from suspicious pancreatic lesions obtained by endoscopic ultrasonography. The most frequent histological diagnosis was PDAC (n=67), followed by IPMN (n=11), benign cyst (n=10), neuroendocrine tumor (n=9) and other malignancies (n=9). The expression of miR-10b was significantly higher in CK19+ cancer cells (6103±2368) and pre-malignant IPMN lesions (3635±1605) compared to CK19+ cells from benign cysts (900±491; p<0.0001 and p<0.0006, respectively).

Based upon this analysis, it was concluded that miR-10b levels are highest in PDAC, intermediate in IPMN, and lowest in benign cysts. These findings indicate that miR-10b could serve as a novel biomarker for the metastatic potential of PDAC and for the malignant transformation of IPMN.

### Example 11

### Involvement of miRNAs in Lung Cancer Biology and Therapy

Detecting early changes in lung carcinogenesis, including key changes in miRNA expression, offers the prospect of intervention to improve clinical outcomes when the potential for substantial clinical benefits exist. In this regard, it is intriguing that altered miRNA expression is detected in sputum. This information can uncover early stages of squamous cell carcinoma (Xing, et al. (2010) Mod. Pathol. 23:1157-64), adenocarcinoma (Yu, et al. (2010) Int. J. Cancer 127:2870-8), and other types of NSCLC (Xie, et al. (2010) Lung Cancer 67:170-6).

Specific miRNA expression profiles can improve lung cancer classification and identify new pharmacological targets. Prior miRNA signatures have been linked to the prognosis of clinical subsets of lung cancers (Yu, et al. (2008) Cancer Cell 13:48-57), which can help to classify this malignancy by precisely defining the miRNA expression profiles that are characteristic of different histopathologic types of lung cancer (Landi, et al. (2010) Clin. Cancer Res. 16:430-41).

Expression profiles of miRNAs can provide prognostic information in lung cancer. Specific miRNA expression patterns can assess survival outcomes for lung cancer patients. For example, a 4-miRNA (miR-486, miR-30d, miR-1, and miR-499) serum signature in NSCLC can predict overall survival (Hu, et al. (2010) J. Clin. Oncol. 28:1721-6). Increased miR-155 and reduced let-7a-2 expression defines an unfavorable survival in pulmonary adenocarcinomas (Yanaihara, et al. (2006) Cancer Cell 9:189-98). Lung cancer patients whose tumors had a 5-miRNA signature (low miR-221 and let-7a, high miR-137, miR-372, and miR-182*) exhibit unfavorable overall and disease-free survivals (Yu, et al. (2008) supra). Recurrence of stage I NSCLC after surgical resection was predicted by miRNA expression profiles (Patnaik, et al. (2010) Cancer Res. 70:36-45).

It is informative to identify lung-cancer-associated miRNAs, which are biomarkers of treatment response or pharmacological targets. Given these data, searches were undertaken to find those miRNAs that would exert tumor-suppressive or oncogenic (oncomir) effects in the lung. Candidate tumor-suppressive miRNAs are those that exhibit a reduced expression in malignant versus adjacent normal lung tissues. Potential oncomirs are defined by overexpression in the malignant versus adjacent normal lung tissues. Consequences of this overexpression include changes in the expression of critical target genes that could confer tumor-suppressive or oncogenic effects of specific miRNAs. Changes in these direct target genes can be used to improve the diagnosis or classification of lung cancers. Some of these species are also therapeutic or chemopreventive targets in the lung, as in the case of miR-31 (Liu, et al. (2010) J. Clin. Invest. 120:1298-309).

Oncomirs in Lung Cancer.

Oncomirs have a higher basal expression in malignant as compared with adjacent normal lung tissues. Some are potential prognostic biomarkers as in miR-92a-2* in SCLC (Ranade, et al. (2010) J. Thorac. Oncol. 5:1273-8) or in the examples of miR-155, miR-130a, let-7f, and miR-30e-3p in NSCLC (Yanaihara, et al. (2006) supra; Wang, et al. (2010) Am. J. Med. Sci. 5:385-8). To date, few candidate oncomirs have had mechanistic validation or identification of the target genes that would exert their oncogenic effects.

One of these oncomirs is miR-21, which plays a key functional role in several cancers (Corsten, et al. (2007) Cancer Res. 67:8994-9000; Asangani, et al. (2008) Oncogene 27:2128-36; Ribas, et al. (2009) Cancer Res. 69:7165-9; Wang, et al. (2009) Cancer Res. 69:8157-65) including lung cancer (Seike, et al. (2009) Proc. Natl. Acad. Sci. USA 106:12085-90). Prior work revealed that miR-21 promotes cellular growth and augments tumor invasion and metastasis (Zhu, et al. (2008) Cell Res. 18:350-9) by reducing the expression of specific sets of target genes that exert tumor-suppressive effects. For example, in K-ras-dependent mouse lung cancers, an increase occurs in miR-21 expression; miR-21 targets multiple negative regulators of the Ras/methyl ethyl ketone/extracellular receptor kinase pathway to promote proliferation by regulating the expression of Spry1, Spry2, Btg2, and Pdcd4 (Zhu, et al. (2008) supra; Hatley, et al. (2010) Cancer Cell 18:282-93; Lu, et al. (2008) Oncogene 27:4373-9). Also, miR-21 inhibits apoptosis by reducing the expression of proapoptotic gene products that include Apaf1, Faslg, Pdcd4, and RhoB (Zhu, et al. (2008) supra; Hatley, et al. (2010) supra). Reducing Pdcd4 expression can increase invasion and metastasis (Lu, et al. (2008) supra).

It has been reported that miR-31 acts as an oncomir in murine and human lung cancers (Liu, et al. (2010) J. Clin. Invest. 120:1298-309). The overexpression of miR-31 and other miRNAs in transgenic cyclin E-driven murine lung cancers implied that a similar miRNA expression profile would occur in human lung cancers (Ma, et al. (2007) Proc. Natl. Acad. USA 104:4089-94). This was the case when human lung cancers (vs. adjacent normal lung tissues) had a similar expression pattern for several miRNAs that were highlighted in previously described transgenic lung cancers (Liu, et al. (2010) supra; Ma, et al. (2007) Proc. Natl. Acad. Sci. USA 104:4089-94). Functional validation was sought for the candidate oncomirs that were overexpressed in most examined murine and human lung cancers.

In a panel of overexpressed miRNAs in murine lung cancers, only a small subset also was augmented in human lung cancers (Liu, et al. (2010) supra). Of those, the basal miR-31 expression level was enhanced in several lung cancer cell lines studied, and an engineered knockdown of miR-31 significantly reduced both cellular growth and colony formation (Liu, et al. (2010) supra). Among the potential target genes uncovered by bioinformatic analysis, several tumor-suppressive species were identified as possible direct miR-31 targets (Liu, et al. (2010) supra). Of these, two were functionally validated as direct miR-31 targets, the large tumor suppressor 2 (LATS2) and PP2A regulatory subunit B alpha isoform (PPP2R2A); an engineered knock-down of LATS2 or PPP2R2A reversed the growth inhibitory effect of miR-31 knock-down (Liu, et al. (2010) supra). This finding revealed a direct link between miRNA- and LATS2 and PPP2R2A expression. Prior work has highlighted miR-31 as a candidate oncomir in head and neck cancer (Liu, et al. (2010) Cancer Res. 70:1635-44). Interestingly, regulating miR-31 expression also affects breast cancer metastasis by regulating the expression of other target genes (Valastyan, et al. (2010) Cancer Res. 70:5147-54; Valastyan, et al. (2009) Cell 137:1032-46). Taken together, these findings underscore that miRNAs exert their functions in cell-, tissue-, and disease-specific contexts. Pharmacologic knock-down of a critical oncogenic miRNA, such as miR-31 in lung cancer, might exert antineoplastic effects (Liu, et al. (2010) supra).

Tumor-Suppressive miRNAs in Lung Cancer.

In contrast to oncomirs, the expression profiles of candidate tumor-suppressive miRNAs are repressed in cancers versus adjacent normal lung tissues. For example, reduced expression of the miRNA let-7 was reported to occur in bronchioloalveolar carcinoma (Inamura, et al. (2007) Lung Cancer 58:392-6) as well as in lung adenocarcinoma (Yanaihara, et al. (2006) supra; Takamizawa, et al. (2004) Cancer Res. 64:3753-6). The miR-146b expression patterns in squamous cell lung cancer predicted a poor clinical outcome, and miR-34a expression was identified as a biomarker for clinical relapse in surgically resected NSCLC (Raponi, et al. (2009) Cancer Res. 69:5776-83; Gallardo, et al. (2009) Carcinogenesis 30:1903-9). Also, miR-34a was inactivated by CpG methylation, which caused transcriptional silencing in lung cancers (Ladygin, et al. (2008) Cell Cycle 7:2591-600). The precise mechanisms through which these miRNAs exert their tumor-suppressive effects remain to be determined.

The miR-34 family is reported as a p53-induced tumor-suppressive miRNA family in diverse types of cancers (He, et al. (2005) Nature 435:828-33; Change, et al. (2008) Nat. Genet. 40:43-50; Corney, et al. (2007) Cancer Res. 67:8433-8; Welch, et al. (2007) Oncogene 26:5017-22; Tazawa, et al. (2007) Proc. Natl. Acad. Sci. USA 104:15472-7; Bommer, et al. (2007) Curr. Biol. 17:1298-307). As a transcription factor, p53 directly induces miR-34 family transcription. Ectopic miR-34 expression can augment apoptosis, cell-cycle arrest, or senescence. The promoter regions of the miR-34 family often are inactivated by CpG methylation (Lodygin, et al. (2008) supra). Repression of the miR-34 family was linked to a resistance to p53-activating agents that can cause apoptotic response to specific chemotherapy treatments (Zenz, et al. (2009) Blood 113:3801-8). Direct miR-34 target genes include CKD4/6 (Fujita, et al. (2008) Biochem. Biophys. Res. Commun. 377:114-9; Sun, et al. (2008) FEBS Let. 582:1564-8; He, et al. (2007) Nature 447:1130-4), c-Myc (Kong, et al. (2008) Proc. Natl. Acad. Sci. USA 105:8866-71; Leucci, et al. (2008) J. Pathol. 216:440-50), CREB (Pigazzi, et al. (2009) Cancer Res. 69:2471-8), SIRT1 (Yamakuchi, et al. (2008) Proc. Natl. Acad. Sci. USA 105:13421-6), cyclin E (Liu, et al. (2009) Clin. Cancer Res. 15:1177-83), and Bcl-2 (Bommer, et al. (2007) supra). It has been shown that miR-34c expression was repressed in both murine and human lung cancers and that ectopic expression of miR-34c significantly reduced lung cancer cell growth by specifically targeting cyclin E for repression (Liu, et al. (2009) Clin. Cancer Res. 15:1177-83). Other highlighted candidate tumor-suppressive miRNAs in lung cancer include miR-145 and miR-152-5p. The expression patterns of oncogenic and tumor-suppressive miRNAs in lung cancers have been confirmed by the use of in situ hybridization (ISH) assays.

The let-7 miRNA family is located in genomic regions that are fragile sites or frequently are deleted in specific cancers (Calin, et al. (2004) Proc. Natl. Acad. Sci. USA 101:2999-3004). Let-7 expression is often repressed in certain types of lung cancers (Yanaihara, et al. (2006) supra; Inamura, et al. (2007) supra; Takamizawa, et al. (2004) supra). Engineered let-7 overexpression can inhibit cancer cell proliferation (Lee & Dutta (2007) Genes Dev. 21:1025-30; Johnson, et al. (2005) Cell 120:635-47). Several let-7 target genes are reported, including K-ras (Johnson, et al. (2005) supra), HMGA2 (Lee & Dutta (2007) supra; Hebert, et al. (2007) Mol. Cancer. 6:5), and c-Myc21 (Koscianska, et al. (2007) BMC Mol. Biol. 8:79), as well as the cell-cycle regulators CDC25A, CDK6, and cyclin D2 (Johnson, et al. (2007) Cancer Res. 67:7713-22). Engineered overexpression of let-7 was achieved in the K-ras-driven murine lung cancer model via adenoviral delivery, which reduced lung cancer formation in vivo (Kumar, et al. (2007) Nat. Genet. 39:673-7). Forced overexpression of tumor-suppressive miRNAs can exert antineoplastic effects in lung cancer (Liu, et al. (2009) supra).

Expression profiles of miRNAs are useful to improve diagnosis and classification as well as to provide clinical prognostic information in lung cancer. miRNA signatures for each histopathologic subtype of lung cancer can be assessed and compared to any changes relative to the normal lung in addition to the clinical features present at diagnosis such as stage, smoking history, age, and gender. Likewise, miRNA profiles can prove informative in predicting the response to chemotherapeutic or targeted therapies. At the same time, miRNA profiles can identify those at high risk for developing lung cancer. This information can guide the use of chemopreventive agents to reduce this clinical risk.

It is known that miRNA profiles exert biological effects by regulating the expression of many target genes. Bioinformatic analysis can predict potential target genes for each miRNA (Lewis, et al. (2003) Cell 115:787-98; Lewis, et al. (2005) Cell 120:15-20). Functional validation of any highlighted target gene can be confirmed by establishing the direct complex formation of the miRNA of interest with the expected mRNA sequence. In these analyses, miRNA profiles could depend on the cell and tissue contexts as well as on the examined physiological or pathophysiological states. It is notable that a change in a single miRNA can lead to compensatory and time-dependent changes in protein (or mRNA) expression profiles within individual cells or tissues (Selbach, et al. (2008) Nature 455:58-63; Baek, et al. (2008) Nature 2008:455:64-71). An examination of any individual or small series of miRNAs may not reflect the complexity of changes in miRNA expression participating in clinical tumor biology. Comprehensive analysis of the functional role of a network of miRNAs and their targets may be needed.

### Example 12

### MiRNA as Biomarkers for Management of Breast Cancer

Altered expression of miRNAs in breast cancer cell lines and clinical breast tissue specimens have been identified using different high-throughput profiling methods (Blenkiron, et al. (2007) Genome Biol. 8:R214; Mattie, et al. (2006) Mol. Cancer. 5:24; Iorio, et al. (2005) Cancer Res. 75:7065-7070; Sempere, et al. (2007) Cancer Res. 67:11612-11620). A small subset of miRNAs linked to breast cancer has been identified in expression profiling experiments on RNA extracted from normal and tumor clinical specimens (Table 8).

TABLE 8

| | | | Role/Special |
|---|---|---|---|
| | | | Features/Potential |
| | | | Clinical |
| miRNA | Change | ISH data | Application(s) |
| Let-7a | Down¹ | Luminal¹ | Growth suppressive |
| | | | and pro-apoptotic; |
| | | | marker for prognosis |
| miR-10b | Down²/Up³ | — | Enhances metastatic |
| | | | potential; marker |
| | | | for recurrence and |
| | | | outcome |
| miR-16 | | — | Pro-apoptotic; |
| | | | marker for prognosis |
| | | | and treatment |
| | | | response |
| miR-17-5p | | — | Pro-apoptotic; |
| | | | marker for prognosis |
| | | | treatment response |
| miR-21 | Up¹ | TAF > ICa > luminal¹ | Proxy marker for |
| | | | PTEN/AKT; treatment |
| | | | response in HER2+ |
| | | | cases |
| miR-27b | | — | Modulates estrogen |
| | | | signaling; marker |
| | | | for treatment |
| | | | response in ER+ |
| | | | cases |
| miR-34a | Down | Luminal > fibroblast | Pro-apoptotic and |
| | | | tumor suppressive; |
| | | | proxy marker for p53 |
| | | | activity status |
| miR-106b | Up | — | Promotes cell |
| | | | proliferation; |
| | | | Prognostic indicator |
| miR-125b | Down | Fibroblast, | Pro-apoptotic, |
| | | myoepithelia | marker for treatment |
| | | | response in HER2+ |
| | | | cases |
| miR-126 | Down | Endothelial | Reduces metastatic |
| | | cells | potential, marker |
| | | | for vascular |
| | | | normalization |
| miR-141 | Up? | Luminal¹ | Modulates ZEB-1, |
| | | | resulting in E- |
| | | | cadherin |
| | | | upregulation; marker |
| | | | for invasion |
| miR-145 | Down¹ | Myoephithelia, | Early detection |
| | | smooth muscle | (myoepithelia); |
| | | | vascular |
| | | | normalization |
| | | | (smooth muscle) |
| miR-155 | Up | — | Prognostic indicator |
| miR-200c | — | — | Modulates ZEB-1, |
| | | | resulting in E- |
| | | | cadherin |
| | | | upregulation; marker |
| | | | for invasion |
| miR-205 | Down¹ | Myoephithelia | Prognostic indicator |
| | | | in basal/ER-PR-HER2 |
| | | | subtype |
| miR-206 | — | — | Proxy marker for ER |
| | | | status and treatment |
| | | | response in ER+ |
| | | | tumors |
| miR-210 | Up | Stroma, luminal | Independent |
| | | | prognostic |
| | | | indicator; proxy |
| | | | marker for hypoxia |
| miR-214 | Up? | Luminal, | Modulates Hedgehog |
| | | fibroblast¹ | signaling |
| miR-221 | Up | — | Promotes cell |
| | | | proliferation; |
| | | | prognostic indicator |
| miR-335 | Down | — | Prevents tumor |
| | | | metastasis; marker |
| | | | for recurrence and |
| | | | outcome |
| miR-373 | Up | — | Increases metastatic |
| | | | potential; marker |
| | | | for recurrence and |
| | | | outcome |
| miR-520c | Up | — | Increases metastatic |
| | | | potential; marker |
| | | | for recurrence and |
| | | | outcome |

Change column refers to miRNA expression change between normal and tumor breast tissue using total RNA extracted from whole tissue using different detection tools (e.g., microarray profiling and/or RT-PCR); accordingly “Down” refers to decrease detection and “Up” to increase detection of miRNA levels in tumor tissue.
Note that only for ISH analysis, the “Change” column refers to expression changes specifically within epithelial cells.
—, to be determined.
¹ISH data from Sempere, et al. (2007) supra.
²Iorio, et al. (2005) supra.
³Ma, et al. (2007) Nature 449: 682-688.

It was contemplated that differential detection could merely reflect cell type heterogeneity among normal and tumor tissues and/or the recruitment of reactive stroma and infiltrating immune cells to the cancerous lesion. To obtain insight into miRNA deregulation in breast cancer, the instant in situ hybridization (ISH) method was implemented to reveal the spatial distribution of miRNA expression in archived formalin-fixed paraffin-embedded (FFPE) specimens, representing normal and tumor tissue from about 100 patient cases (Sempere, et al. (2007) supra).

Fluorescent Substrate-Based ISH Procedure on Automated Staining Station.

Tissue samples (4 μm slices) were deparaffinized in xylenes, re-hydrated in an ethanol dilution series, and mounted on positively-charged barrier frame slides. The tissue was digested with proteinase K to facilitate probe penetration and exposure of miRNA species (thought to be tightly associated with proteins of the miRISC). To minimize non-specific binding based on charge interactions, tissue was briefly subject to an acetylation reaction with an acetic anhydride/triethanolamine solution. Then, the tissue was pre-hybridized at the hybridization temperature (Thyb: 25° C. below calculated Tₘ of the probe) for 30 minutes in hybridization solution, which was composed of 50% deionized formamide, 5× Sodium chloride-Sodium citrate buffer (SSC), 1×Denhardt's solution, 500 μg/ml yeast tRNA, 0.01% TWEEN. Subsequently, this hybridization solution was replaced, with hybridization solution containing 10 pmol of the hapten-labeled LNA probe and incubated for 90 minutes. Tissue slides were washed three times for 10 minutes in SSC buffer at the established stringency of SSC (between 1× to 0.2×) at Thyb. At this point, slides were loaded onto the i6000 staining machine, which automatically dispensed 400 μL per slide. The machine washed the slides with phosphate-buffered saline and 0.01% TWEEN (PBST) solution. Slides were subsequently treated with 3% H₂O₂ to inactivate endogenous peroxidase and blocked with bovine serum albumin (BSA) to minimize non-specific binding of the rabbit anti-hapten/HRP antibody with or without further amplification with anti-rabbit/HRP secondary antibody. Following antibody incubation and washes, the tyramine-conjugated fluorochrome was applied to the slide and the TSA reaction proceeded for 10-30 minutes. Finally, slides were profusely washed with PBST and were ready to be mounted on anti-fading PROLONG gold solution (Invitrogen) with or without DAPI (for nuclear counterstaining) and examined under fluorescence microscopy, or kept in PBST for further processing.

miRNA Expression in Breast Cancer Samples.

Lower detection of miR-451 levels in whole tumor tissue compared to normal by microarray profiling did not confirm to be etiologically relevant, but rather it indirectly reflected architectural changes of the tumor-associated vasculature; miR-451 was predominantly expressed in mature erythrocytes as determined by ISH and independently confirmed by functional assays of hematopoietic cell differentiation (Zhan, et al. (2007) Exp. Hematol. 35:1015-1025; Rathjen, et al. (2006) FEBS Lett. 580:5185-5188). However, a subset of miRNAs whose expression was altered within distinct subpopulations of mammary epithelial cells was found, thereby validating and refining expression profiling results. miR-145 and miR-205 expression was restricted to myoepithelial cells in normal epithelial structures, whereas their expression was reduced or completely eliminated in matching tumor specimens. Conversely, expression of let-7a, miR-21, miR-141, miR-214 was detected at varying levels predominantly within luminal epithelial cells in normal tissue. Let-7a expression was consistently decreased within cancer cells, while miR-21 expression was frequently increased within cancer cells, but also in tumor-associated fibroblasts. Expression of miR-141 and miR-214 exhibited a more complex pattern requiring a larger sample size to determine whether their expression changes are associated with any clinical parameters. The most significant associations that were identified were early manifestation of altered miR-145 expression in carcinoma in situ lesions adjacent to invasive carcinoma and thus presumed pre-invasive; and high miR-205 expression correlates with favorable clinical outcome in ER-PR-HER2-cases, although in a limited sample of 20 cases. This indicates the potential clinical application of miR-145 as novel biomarker for early detection of malignancy and disease progression from non-invasive to pre-invasive lesions, and miR-205 as prognostic indicator for the aggressive ER-PRHER2-(basal) subtype.

Different experimental strategies have been followed in a basic research setting to understand the functional contribution of miRNAs to specific processes during breast cancer progression and metastasis, such as cell migration and invasion. These elegant experiments in cell culture systems and mouse models of breast cancer have suggested detailed molecular mechanisms of miRNA-mediated regulation (Bhaumik, et al. (2008) Oncogene 27:5643-5647; Burk, et al. (2008) EMBO Rep. 9(6):582-9; Gregory, et al. (2008) Nat. Cell Biol. 10:593-601; Frankel, et al. (2008) J. Biol. Chem. 283:1026-1033; Huang, et al. (2008) Nat. Cell Biol. 10:202-210; Tavazoie, et al. (2008) Nature 451:147-152; Ivanovska, et al. (2008) Mol. Cell Biol. 28:2167-2174; Ma, et al. (2007) Nature 449:682-688). However, only weak clinical associations have been observed or suggested. For example, miR-10b was mechanistically linked to the initiation of distant metastasis in breast cancer cell lines (Ma, et al. (2007) supra). The initial report suggested that elevated expression of miR-10b as determined by RT-PCR in whole tissue primary tumors of patients with metastatic disease (n=18) compared to metastasis-free (n=5) was predictive of recurrence and poor clinical outcome (Ma, et al. (2007) supra). An independent study determined expression of miR-10b using a similar technical approach in a much larger sample of patient cases (n=219) and found no correlation between miR-10b expression and number of involved lymph nodes (indicative metastatic potential), distant metastases, recurrence-free survival or distant-relapse-free survival (Gee, et al. (008) Nature 455:E8-E9). In response to these findings, the authors of the original study proposed that miR-10b expression may be restricted to and be etiologically relevant in cells at advancing edge of the tumor and lamented that whole tissue profiling may confound the signal of miRNAs such as miR-10b expressed in a restricted number of cells (Ma, et al. (2007) Nature 455:E8-E9).

Proxy Indicator of Tumor Suppressive Pathway.

miR-34a was predominantly expressed in luminal epithelial cells and its expression was markedly reduced in carcinoma in situ (CIS) and invasive carcinoma (Ica) lesions. Therefore, miR-34a could be useful as an indicator of progression from non-invasive to preinvasive disease within a different epithelial subpopulation. Moreover, miR-34a is of particular interest given the well-established transcriptional activation of miR-34 by tumor suppressor gene p53 and its pro-apoptotic and anti-mitogenic role in cancer cells (He, et al. (2007) Nature 435:828-833). p53 expression detected by IHC and/or p53 sequencing analysis may not necessarily reflect loss of p53 activity status, which is associated with poor prognosis (Troester, et al. (2006) BMC Cancer 6:276). Since inactivation of p53 is not always associated with loss of p53 expression, miR-34a levels could inform by proxy of p53 activity status. Thus, miR-34 and other miRNAs could serve as powerful indicators of the status of tumor suppressive or oncogenic pathways.

Prognostic Indicator.

miR-210 was detected in epithelial cells and stroma in normal breast tissue, and expression of miR-210 persisted in matched CIS and ICa lesions. miR-210 has been suggested as an independent prognostic indicator and upregulation of miR-210 expression correlates with hypoxia (Camps, et al. 92008) Clin. Cancer Res. 14:1340-1348).

Predictor of Treatment Response.

miR-125b accumulated at higher levels in myoepithelial cells and surrounding fibroblasts in normal breast tissue.

Experiments in HER2-overexpressing SK-BR-3 cells indicated that miR-125 regulated HER2 and HER3 expression via binding to complementary sites on their mRNAs (Scott, et al. (2006) J. Biol. Chem. 282(2):1479-86), and inverse correlation of miR-125 expression and that of HER2 has been observed in clinical breast cancer specimens (Mattie, et al. (2006) supra). Therefore, the cancer cells with higher levels of miR-125b could be more susceptible to herceptin treatment since miR-125b and herceptin may act cooperatively to disrupt HER2 signaling and thus miR-125b expression could be useful as predictor of treatment response in HER2+ cases.

Vascular Normalization.

It was observed that miR-126 expression was confined to endothelial cells in normal breast tissue. Similarly, miR-126 has been reported to be predominantly expressed in endothelial cell lining the blood vessels by whole mount ISH on zebrafish and mouse embryos (Kloosterman, et al. (2006) Nat. Methods 3:27-29; Wienholds, et al. (2005) Science 309(5732):310-1). Unexpectedly, a decrease of miR-126 expression was observed in tumor-associated vasculature. Endoglin or CD105 is a cell surface glycoprotein and functions as a co-receptor for TGF-β and it is upregulated in endothelial cells of neovasculature (Duff, et al. (2003) FEBS Lett. 17:984-992). A high number of CD105-positive blood vessels and high levels of CD105 in serum are associated with poor prognosis and decreased treatment response (Beresford, et al. (2006) Br. J. Cancer 95:1683-88; Vo, et al. (2008) Breast Cancer Res. Treat. 119(3):767-71; Kumar, et al. (1999) Cancer Res. 59:856-861). Codetection experiments of miR-126, CD31 (general endothelial cell marker) and CD105 indicated that miR-126 and CD105 exhibited opposite expression patterns in normal and tumor-associated blood vessels. Thus, miR-126 may be a useful marker to interpret CD105 staining and refine the assessment of vascular normalization. This also points to the fact that miRNA changes could be clinically informative in cell types other than the cancer cells per se.

microRNA Markers as Predictors of Complete Response.

Mutation status of PTEN and HER2/HERS heterodimerization appear to be important factors for cancer cells to develop resistance to herceptin treatment (Menendez & Lupu (2007) Breast Cancer Res. 9:111; Robinson, et al. (2006) Clin. Breast Cancer 7:254-261; Berns, et al. (2007) Cancer Cell 12:395-402; Fujita, et al. (2006) Br. J. Cancer 94:247-252; Nagata, et al. (2004) Cancer Cell 6:117-121; Sergina, et al. (2007) Nature 445:437-441). miR-21 and miR-125 negatively regulate expression of PTEN (Meng, et al. (2007) Gastroenterology 133:647-658) and HER2/HER3 (Scott, et al. (2006) supra) in breast cancer cell lines, respectively. Thus, miR-21/PTEN and miR-125/HER-2/3 interactions are operational in tumors as suggested by these in vitro experiments, and could notably affect the efficacy of herceptin treatment. Thus, these miRNAs are informative for HER2+ tumors. It is believed that miR-125 could cooperate with herceptin to inhibit HER2/3 signaling, whereas miR-21 would oppose herceptin action by inhibiting PTEN, the natural suppressor of PI3K/AKT kinase pathway downstream of HER2 signaling. Thus, HER2+ tumors with high levels of miR-125 and/or low levels of miR-21 would be more sensitive to herceptin treatment. miR-27b and miR-206 have been implicated in the regulation of estrogen signaling; miR-206 binds and represses ERα mRNA in breast cancer cell lines (Adams, et al. (2007) Mol. Endocrinol. 21:1132-1147) and miR-27b indirectly affects the metabolism of 17-β-estradiol by repressing cytochrome P4501B1 (CYP1B1) (Tsuchiya, et al. 92006) Cancer Res. 66:9090-9098). Thus, these miRNAs could be informative for ER2+ tumors. It is believed that these miRNAs could cooperate with tamoxifen or anti-estrogenic treatments to dampen ER signaling. Thus, ER+ tumors with higher levels of miR-206 and/or miR-27b would be more sensitive to anti-estrogenic treatments. Other miRNAs such as let-7a, miR-141 and miR-221 have also been implicated in the regulation of etiological relevant proteins for breast cancer such as c-Myc (Akao, et al. (2006) Biol. Pharm. Bull. 29:903-6; Sampson, et al. (2007) Cancer Res. 67:9762-9770), E-cadherin (Hurteau, et al. (2007) supra), and p27 (Gillies & Lorimer (2007) Cell Cycle 6:2005-2009), respectively.

miRNA Markers as Indicators of Clinical Outcome.

Pathological complete response is the best surrogate indicator to estimate long term clinical outcome (Chow, et al. (2006) Biomed. Pharmacother. 60:259-62). Patients with pathological partial response represent a mixed population; some will rapidly succumb to the disease, while others will have a long term disease-free survival. It is contemplated that miR-126 is useful as a marker of vascular normalization in patients with a partial response to treatment to stratify cases with respect to disease-free survival. In this respect, other miRNAs could also serve as surrogate markers of “epithelial normalization”. More precisely, high levels of pro-apoptotic miRNAs miR-16 and miR-17-5p and/or low levels of proliferation could be indicators of a favorable outcome.

These data indicate the involvement of a discrete number of miRNAs in the initiation and progression of breast cancer (Table 8). The instant in situ hybridization (ISH) method, however, provided a more direct and informative assessment of how altered miRNA expression contributes to breast carcinogenesis when compared to miRNA expression profiling in whole tissue specimens.

Multi-Color Fluorescent Codetection of miRNA and Protein Markers.

Sequential TSA reactions were carried out on the same tissue specimens. HRP enzymes from previous TSA reactions were inactivated by incubation with 3% H₂O₂ before proceeding to the next round of antibody binding and TSA reactions. Thus, several miRNA species and/or protein markers could be codetected using different fluorochrome for each TSA reaction. Four different tyramine-conjugated fluorochrome substrates were generated for the TSA reaction: AMCA (blue), FITC (green), Rhodamine (red) and ALEXA-647 (infrared). Although, FITC was primarily used as hapten for the miRNA probes, other commercially available moieties such as digoxigenin and biotin could be used to end-labeled the LNA probes.

Table 9 provides a list of protein markers, whose codetection with specific miRNAs will be relevant for the diagnosis, prognosis, treatment, and management of breast cancer.

TABLE 9

| | | | Role/Special |
|---|---|---|---|
| | | | Features/Clinical |
| Protein | Cell Marker | ISH/IHC | Application |
| ER | Luminal | Yes + AR1 | Diagnostic and |
| | | | treatment selection- |
| | | | Test miR-206/ER |
| | | | interaction |
| CK-7 | Luminal | Yes | CK7/CK20 positivity for |
| | | | discriminating ICa of |
| | | | unknown site of origin |
| CK-20 | Non-Breast | — | CK7/CK20 positivity for |
| | Epithelia | | discriminating ICa of |
| | | | unknown site of origin |
| CK-19 | Luminal | Yes | General ICa cell marker |
| CK-5/6 | Myoepithelia | Yes + AR1 | Basal subtype |
| CK-14 | Myoepithelia | Yes + AR2 | Basal subtype |
| ΔN-P63 | Stem Cells | — | Basal subtype |
| P63 | Variable | — | Basal subtype |
| E-cadherin | Epithelia | Yes + AR1 | Discriminates IDCa from |
| | | | ILCa- |
| | | | Test miR-141~miR- |
| | | | 200c/E-cad regulatory |
| | | | loop |
| Vimentin | Stroma | Yes | Genera fibroblast |
| | | | marker, epithelial- |
| | | | mesenchymal transition |
| CD31 | Endothelia | Yes + AR1 | General vasculature |
| | | | marker, vascular |
| | | | normalization |
| CD105 | Endothelia* | Yes + AR1 | *Specifically |
| | | | identifies tumor- |
| | | | associated de novo |
| | | | vasculature |
| HER2 | Variable | Yes + AR1 | Treatment response |
| | | | (herceptin)- |
| | | | Test miR-125/HER2 |
| PTEN | Variable | Yes + AR1 | Treatment response |
| | | | (herceptin)- |
| | | | Test miR-21/PTEN |
| | | | interaction |
| pAKT | Variable | Yes | Treatment response |
| | | | (herceptin) |
| P16 | Variable | Yes + AR1 | Proliferation index, |
| | | | multivariate analysis |
| | | | with COX-2, P16 for |
| | | | progression |
| P53 | Variable | Yes + AR1 | Prognostic indicator- |
| | | | Test miR-34 as proxy |
| | | | of p53 activity status |
| Ki-67 | Variable | Yes + AR1 | Proliferation index, |
| | | | multivariate analysis |
| | | | with COX-2, P16 for |
| | | | progression |
| COX-2 | Variable | Yes + AR1 | Progression, |
| | | | multivariate analysis |
| | | | with COX-2, P16 for |
| | | | progression |

Yes, protein detection is compatible with previous ISH procedure without further treatment or followed by heat-induced epitope retrieval in decloaking chamber (Biocare Medical) with citrate (Vecta Laboratories; AR1) or reveal (Biocare Medial; AR2).
—, to be determined.
ICa: Invasive carcinoma,
IDCa: invasive ductal carcinoma,
ILCa: invasive lobular carcinoma.

This list includes cell-type specific markers for colocalization studies and proteins with clinical value for different aspects of breast cancer management. Multivariate analysis of Ki-67/p16/COX-2 is indicative a disease progression as assayed by IHC on pure DCIS specimens (Gauthier, et al. (2007) Cancer Cell 12:479-491). To determine whether combinations of these markers is a more powerful predictor for disease progression, the predictive value of, e.g., Ki-67/p16/COX-2 and miRNAs is compared. Some of these proteins have been shown to be regulated by miRNAs (e.g., in Table 8) via translational repression or relay the action of some of these proteins. Clinical validation of these miRNA/protein interactions could be more informative than separate assessment of individual markers. This includes the study of E-cadherin and miR-141 as indicators of invasion and prognosis; loss of E-cadherin weakens epithelial cell-to-cell junctions and facilitates an epithelial to mesenchymal transition, which leads to cell invasion and metastasis. miR-141 has been implicated in the maintenance of E-cadherin expression and thus epithelial identity via inhibition zinc-finger transcriptional factor ZEB1, which in turn repress E-cadherin gene expression (Burk, et al. (2008) supra; Gregory, et al. (2008) Nat. Cell Biol. 10:593-601; Hurteau, et al. (2007) Cancer Res. 67:7972-76).

To demonstrate codetection, analysis of miRNAs and cytokeratins that were specifically expressed within different epithelial cell subpopulations such as myoepithelial layer (e.g., CK5/6, CK14) and luminal epithelia (CK7, CK19) was conducted. While anti-CK7 and CK19 indiscriminately stain breast adenocarcinomas ascribed to any of the major tumor subtypes, CK5/6 and CK14 among other protein markers are widely used to diagnose the aggressive basal subtype. Thus, codetecting miRNA expression with cytokeratins, but also with other markers that specifically labeled endothelial cells (e.g., CD-31) or fibroblasts/stroma (e.g., vimentin) greatly assists in determining the cell-type distribution of miRNA expression in breast tissue and interpreting changes of miRNA in different tumor subtypes.

Additional codetections experiments include the codetection of: (a) a miRNA and protein levels of its target gene (miR-21 and PTEN, miR-125b and HER2, miR-206 and ER, miR-221 and ER) to demonstrate the mechanistic interactions engaged in breast cancer; (b) miR-34a and P53 to demonstrate whether miR-34a could inform by proxy of p53 activity status since inactivation of p53 is an indicator of poor prognosis; (c) let-7a and Ki-67 and miR-210 and Ki-67 to demonstrate a negative and positive correlation of miRNA expression, respectively, with cell proliferation as indicator of cell aggressiveness; and (d) miR-205 and E-cadherin and metastatic miRNAs (miR-10b, miR-373 and miR-520c) and E-cadherin to demonstrate a negative and positive correlation of miRNA expression, respectively, with an EMT phenotype (absent/low E-cadherin levels) as indicator of metastatic potential.

### Example 13

### Automation of miRNA and Protein Marker Detection

An automated pipeline from codetection of miRNA and protein markers on high-density tissue microarrays (TMAs) can employ an XMATRX® staining station (BioGenex), high-resolution image acquisition using a multi-spectral VECTRA™ system (CRi), and computer-assisted image analysis using INFORM™ software package (CRi). Major advantages of this fully automated approach include: increase experimental reproducibility, increase multiplexing capability up to eight independent markers, and much faster turn-around time from assay to data analysis.

XMATRX® is an FDA-approved fully automated staining station to perform IHC, ISH and FISH assays. The XMATRX® performs all steps from slide baking to final anti-fading media mounting and coverslipping. XMATRX® automatically places and removes coverslips on top of the slides during critical steps, including incubations at high temperature during miRNA probe hybridization (50° C.) and HIER (98° C.) and incubations with small volume (30-100 μL) to minimize cost of expensive reagents such as miRNA probes, primary antibodies and fluorochrome substrates. The XMATRX® instrument can run up to 40 different slides per experiment; each slide is placed on a separate heat block, allowing independent temperature conditions and programs. This feature is ideal for high-throughput optimization of miRNA probe conditions. XMATRX® also increases the technical reproducibility of the instant combined ISH/IHC methodology since time- and temperature-sensitive steps of Proteinase K digestion and HIER are standardized by automation.

The VECTRA™ system is multi-spectral microscope capable of both fluorescence and bright field imaging. VECTRA™ system provides a more quantitative, more efficient and faster approach to acquire image data from whole tissue and TMA (tissue microarray) slides. VECTRA™ uses a unique liquid-crystal tunable system that allows the separation of fluorochromes with similar emission spectra, and also removes background autofluorescence. With this VECTRA™ system, codetect of up to eight signals is possible including fluorescent stains: DAPI (nuclear counterstaining for cell segmentation), AMCA, FITC, Rhodamine, Dylight594, Dylight649, and Dylight680, and chromogenic stains: DAB, BCIP/NBT and/or fast red (nuclear fast red as an alternative for counterstaining).

The image analysis software package INFORM™ is the companion of the VECTRA™ system. INFORM™ identifies individual cells by detecting a nuclear marker (e.g., DAPI) and applying an algorithm for cell segmentation (essentially a nuclear and cytoplasmic outline based on nuclear size and shape, and arrangement and proximity of cells). After cell segmentation, INFORM™ quantifies the intensity of each individual signal and keeps track of mean intensity (and other parameters) for each defined cell. In order to quantify miRNA expression in individual cells within specific cell compartments, a cell type-specific marker (e.g., CK19 for epithelial/cancer cells) is used to create a mask that highlights the marker positive compartment (e.g., epithelial cells) from the marker negative compartment (e.g., non-epithelial). A similar analysis can be performed to assess regulatory interactions in clinical specimens between specific miRNAs and their target genes.

Not to be limited by this exemplary disclosure, there are other automated staining stations which could be used in combination with the instant method. These include AUTOSTAINER 720™ by (Thermo) Lab Vision, BENCHMARK LT™ by Ventana Med. Systems, BOND™ by Vision Biosystems (Leica), AUTOSTAINER UNIVERSAL™ by Dako, which are amenable and programmable to perform multi-color codetection of miRNA, RNA and protein markers. Similarly, there are other image capturing and analyzing platforms such as ScanScope FL system and accompanying ImageScope software package by Aperio, Pannoramic Fluorescent Scanner by 3DHistech, and VS110 for Fluorescence by Olympus, and custom-designable MicroVigene software package by VigeneTech that are amenable and programmable to perform data acquisition and analysis of this present multi-color combined ISH/IHC assay.

Preliminary results of fully automated codetection of miRNAs, U6 and proteins using XMATRX® and INFORM™ was conducted. For example, miR-126, U6, CK19 and PDCD-4 were codetected in a breast cancer sample. It was observed that intense cytoplasmic staining of CK19 highlighted normal luminal epithelial cells and carcinoma islands, whereas nuclear staining of small nuclear RNA U6 was present in all cell types. Cytoplasmic-enriched staining of miR-126 was confined to endothelial cells and was more apparent in the normal vasculature. PDCD-4 expression was markedly reduced in carcinoma cells compared to normal epithelia and other cell types.

Using this fully automated system in conjunction with the instant method, the association of other miRNAs with cancer can be assessed. For the purposes herein, “cancer-associated miRNAs” refers to those miRNAs whose levels change between normal and tumor tissue in carcinomas of the breast, colon, lung and/or pancreas and those miRNAs which have been shown to exhibit oncogenic or tumor suppressive in cell line systems and/or mouse models of these solid tumors. miRNAs can be grouped into broad categories with respect to their reported tumor suppressive and oncogenic functions. Highly similar miRNA family members (e.g., let-7a, let-7b, let-7f with one or two nucleotides differences in the mature sequence) are listed as a single entry (e.g., let-7s). The tumor suppressive group includes those miRNAs with anti-proliferative, pro-apoptotic and/or anti-metastatic properties: let-7s, miR-15, miR-16, miR-26b, miR-27b, miR-34s, miR-125b, miR-141, miR-200s, miR-205, and miR-335. The oncogenic group includes those miRNAs with pro-mitogenic, anti-apoptotic and/or pro-metastatic properties: miR-10b, miR-17, miR-18, miR-19, miR-20, miR-21, miR-23s, miR-31, miR-106b, miR-206, miR-214, miR-221, miR-222, miR-373, and miR-520c. Other cancer-associated miRNAs categorized by changes in RNA levels in tumor tissues are listed in Table 10. These include miRNAs detected at lower levels in tumor tissue: miR-126, miR-143, and miR-145 and miRNAs detected at higher levels in tumor tissue: miR-107, miR-132, miR-155, miR-196s, miR-210, and miR-213. Finally, a miscellaneous group of miRNAs, which may play a role in cancer via modulation of immune system are also identified and include the miR-29 family, miR-142, miR-146, and miR-181.

TABLE 10

| | | General | Specialized | |
|---|---|---|---|---|
| Source | | Cell-Type | Cell-Type | Functional |
| Cell | miRNA | Marker | Marker | Marker |
| Epithelia | let-7a↓, | CK19 | CK5/6, CK7, | p53, |
| | miR-21↑, | | CK14, CK20 | Ki-67, |
| | miR-34a↓, | | | E-cadherin |
| | miR-125b↓, | | | |
| | miR-141↓, | | | |
| | miR-205↓ | | | |
| Smooth | miR-143↓, | smooth | | |
| Muscle | miR-145↓, | muscle | | |
| | miR-206 | actin | | |
| Fibroblast | miR-21↑^{#}, | Vimentin | | |
| | miR-125b↓ | | | |
| Endothelia | miR-126↓, | CD31 | CD105 | |
| | miR-132↑^{#}, | | | |
| | miR-210↑ | | | |
| Immune | miR-29b, | CD45 | CD3, CD11b, | CD45RA, |
| Cells | miR-146, | | CD19, CD68 | CD45RO |
| | miR-155↑, | | | |
| | miR-181 | | | |

Different source cells of cancer-associated miRNAs and protein markers are listed. Cell type(s) of miRNA expression and/or activity as determined by ISH detection assay (miR denotes exclusive expression), expression profiling assays, in vitro cell line assays and/or knockout mouse studies.
^{#}indicates that changes were observed in tumor-associated fibroblast or tumor-associated vasculature. Arrows indicate general trend of increase (↑) or decrease (↓) miRNA levels in whole tumor tissue specimens from breast, colon, lung, and pancreas.

Given that the combined ISH/IHC assay herein can use three or four fluorescent stains for detection of miRNA, U6 snRNA, cell type protein markers and other protein markers, up to three more protein markers can be codetected. These proteins include the product of target genes, regulators of miRNA expression and proteins that reflect a biological output downstream of the miRNA-regulated event(s) (Table 11). For example miR-34 family members (miR-34a,b,c) are transcriptionally activated by the p53 tumor suppressor gene in response to DNA damage and mitogenic signals. Consistent with a tumor suppressive role, miR-34s were detected at lower levels in breast, lung and other solid tumors by expression profiling analyses in whole tissue biopsies. Co-staining with CK7 (epithelial marker), p53 (tumor suppressor gene) and Ki-67 (proliferation marker) protein markers indicated lower accumulation of miR-34a levels within malignant cells in lung and breast cancer compared to normal epithelial cells as well as normal and reactive stroma. Detection of p53 expression by IHC was suggestive of discrete or point mutations that interfered with p53 tumor suppressive functions, while negative IHC staining was not informative because it could not distinguish other mutated forms or chromosomal deletion of p53 from wild-type and functional p53 gene. Expression of other miRNAs is also activated by p53 (Table 11). Codetection of these miRNAs and p53 may identify different molecular subtypes of functional, null or mutated p53. Thus, expression of miR-34a or other miRNAs could inform by proxy of p53 activity status and provide a better prognostic indicator than a p53-based IHC assay alone.

Furthermore, miR-21 is expressed at high levels within cancer cells (lung and pancreatic cancer) and tumor-associated fibroblasts (TAFs) (breast and colorectal cancer) compared to matched normal tissues (Sempere, et al. (2007) supra), as supported by co-staining with the mesenchymal markers vimentin and smooth muscle actin. In vitro studies indicated that miR-21 inhibits the expression of tumor suppressive PTEN. Therefore, the coexpression of miR-21 and PTEN protein levels was analyzed in carcinoma lesions of the breast, colon, lung, pancreas and prostate tissue. These results indicate a regulatory interaction, especially in lung adenocarcinomas.

TABLE 11

| miRNA | Target Gene | Pathway-related |
|---|---|---|
| Let-7a | KRAS, c-Myc | Ki-67 (proliferation readout) |
| miR-10b | HOXD10 | Twist (direct activator) |
| miR-16 | BCL-2 | Cleaved caspase-3 |
| | | (apoptosis readout) |
| miR-19 | PTEN | c-Myc (direct activator) |
| miR-21 | PTEN, PDCD-4 | HER-2 (direct activator) |
| miR-34a | CDK4/6 | P53 (direct activator) |
| miR106b | P21 | c-Myc (direct activator) |
| miR125b | HER2/3 | P53 (direct activator) |
| miR-141 | ZEB-1/2 | E-cadherin (invasion readout) |
| miR-221 | P21, P27 | Ki-67 (proliferation readout) |

These results underscore the feasibility of codetecting a miRNA and protein levels of target gene(s) on the same tissue section.

### Example 14

### Codetection of Markers in Cancer Tissue Specimens

Codetection of miRNAs and Other Non-Coding RNAs by ISH in Bladder Cancer Tissue Specimens.

Representative tissue cores of bladder cancer tissue microarrays were analyzed. Expression of a miRNA (miR-21 or miR-34a) and control non-coding RNA (snRNA U6 or 18S rRNA) was detected by consecutive rounds of TSA reactions with green and blue fluorescent substrates. Tissue sections were counterstained with nuclear marker DAPI (blue).

Codetection of miRNAs and Other Non-Coding RNAs by ISH in Lung Cancer Tissue Specimens.

Representative tissue cores of lung cancer tissue microarrays were analyzed. Expression of miR-31 and control non-coding RNA snRNA U6 was detected by consecutive rounds of TSA reactions with green and blue fluorescent substrates. Tissue sections were counterstained with nuclear marker DAPI (blue). Tissue cores of this tissue microarrays were scored based on overall intensity of signal of miR-31 in the cancer cells (0=No signal; 1=low or intermediate signal; 2=high signal).

Codetection of miRNA and Protein Markers in Formalin-Fixed Paraffin-Embedded Clinical Specimens of Breast Cancer.

Composite images were generated to depict the expression of miRNAs and proteins in breast cancer tissue. Membranous staining of E-cadherin highlighted carcinoma islands. Elevated expression of TP53 within carcinoma cells was an indication of the mutated state of the cells. Cytoplasmic-enriched staining of miR-21 was most apparent in the reactive stroma around carcinoma islands.

Codetection of miRNA and Protein Markers in Formalin-Fixed Paraffin-Embedded Clinical Specimens of Breast Cancer.

Composite images were generated to depict the expression of microRNA, non-coding abundant RNA and proteins in normal and cancerous breast tissue. Intense cytoplasmic staining of CK19 highlighted normal luminal epithelial cells and carcinoma islands. Nuclear staining of small nuclear RNA U6 was present in all cell types in the mammary tissue (i.e., epithelial, endothelial, stromal and immune cells). Cytoplasmic-enriched staining of miR-126 was confined to endothelial cells and was more apparent in the normal vasculature. PDCD-4 expression was markedly reduced in carcinoma cells compared to normal epithelia and other cell types.

Combination of Chromogenic and Fluorescent Stainings for Codetection of miRNA and Protein Markers in Formalin-Fixed Paraffin-Embedded Clinical Specimens of Breast Cancer.

Composite image were generated to depict expression of miR-21 and CK19 and nuclei counterstaining with hematoxylin. CK19 staining highlighted carcinoma islands. Elevated expression of TP53 within carcinoma cells was indicative of the mutated state. Cytoplasmic-enriched staining of miR-21 was evident showing high levels of expression in cancer cells and at even higher levels in the reactive stroma.

Spatial Characterization of miRNA Expression Profiles in Murine and Human Lung Tissues.

Representative ISH assay results generated using LNA-modified probes against candidate tumor-suppressive or oncogenic miRNAs. These assays were performed using formalin-fixed, paraffin-embedded normal or malignant lung tissues. The results showed the miRNA expression patterns of the indicated miRNAs in age-matched, nontransgenic sibling controls (normal lung tissue) and in a cyclin E-driven mouse model of lung cancer. ISH analyses indicated a predominant bronchial epithelial cell expression pattern for miR-34c, as well as for the expression of miR-145 in epithelial cells, although miR-145 also was expressed in the smooth muscle cells of the lamina muscularis of the mucosa and vasculature. In contrast, miR-126 expression was expressed in endothelial cells and by this profile was viewed as having a lower priority for miRNA functional assessment than either miR-34c or miR-145. ISH expression patterns for miR-31 and for the 18S ribosomal RNA (as control for RNA integrity) in paired human normal lung tissue versus adjacent lung cancer (adenocarcinoma) were generated. This ISH analysis confirmed augmented miR-31 expression (relative to adjacent normal lung) within both murine and human lung cancers. Representative hematoxylin and eosin stained lung tissues were also prepared.

Codetection of microRNA and Protein Markers in Formalin-Fixed Paraffin-Embedded Clinical Specimens of Colon Cancer.

Composite images were generated to depict expression of microRNA and protein markers in cancerous colonic tissue. Intense cytoplasmic staining of CD68 highlighted macrophages infiltrating the tumor. Cytoplasmic-enriched staining of miR-155 was confined to a subpopulation of CD68⁻ immune cells.

Codetection of microRNA and Protein Markers in Formalin-Fixed Paraffin-Embedded Clinical Specimens of Lung Cancer.

Composite images were generated to depict expression of miR-155 and cell type-specific markers in cancerous lung tissue. CD45RO and CD45RA staining indicated different subpopulations of immune cells. CK-7 staining highlighted the carcinoma compartment in this cancerous lesion. Cytoplasmic-enriched staining of miR-155 was confined to a subpopulation of CD45RO⁻/CD45RA⁻ immune cells.

Codetection of Multiple Protein Markers in Formalin-Fixed Paraffin-Embedded Clinical Specimens of Breast Cancer.

Composite images were generated to depict expression of CD markers specific of subpopulations of immune cells and cytokeratin 19. CK19 staining highlighted carcinoma islands. Membranous staining of CD3 identified T cell populations. Membranous staining of CD68 identified macrophages. These combinations of markers enable characterization of miRNA expression of, e.g., miR-29, miR-146 and miR-155 in immune cell infiltrates.

Codetection of Multiple Protein Markers in Formalin-Fixed Paraffin-Embedded Clinical Specimens of Breast and Colon Cancer.

Composite images were generated to depict expression of CD markers specific of subpopulations of immune cells, which exhibit different infiltration patterns in breast and colon cancer. Membranous staining of CD3 identified T cell populations. Cytoplasmic-enriched staining of MPO identified neutrophils/myeloid immune cell lineages. These combinations of markers enable characterization of miRNA expression such of, e.g., miR-29, miR-146 and miR-155 in immune cell infiltrates.

Combination of Chromogenic and Fluorescent Staining to Codetect HER2Gene Locus and HER2Expression by iFISH Formalin-Fixed Paraffin-Embedded Clinical Specimens of Breast and Colon Cancer.

HRP-mediated chromogenic staining was used to detect HER2 expression. FISH assay followed to detect centromeric region of chromosome 17 and HER2 gene locus. IHC and FISH results indicated that this tumor did not overexpress HER2 and lacked a chromosomal amplification of HER2 locus. This data indicated that the patient was HER2− and would not be eligible for HER2 targeted therapy.

Codetection of microRNA and Protein Markers in Formalin-Fixed Paraffin-Embedded Clinical Specimens of Breast Cancer.

Composite images were generated to depict expression of cytokeratin markers and miRNA in breast cancer. Myoepithelial staining was evident for miR-205 and CK14, whereas CK19 exhibited luminal staining.

Codetection of microRNA and Protein Markers in Formalin-Fixed Paraffin-Embedded Clinical Specimens of Colon Cancer.

Composite images were generated to depict expression of marker protein and miRNA in colon cancer. miR-126 and CD31 stained blood vessels, whereas CK20 exhibited cancer cell staining.

Codetection of microRNA and Protein Markers in Formalin-Fixed Paraffin-Embedded Clinical Specimens of Lung Cancer.

Composite images were generated to depict expression of marker protein and miRNA in lung cancer. miR-34a stained stroma, whereas CK7 and P53 exhibited cancer cell staining.

Codetection of microRNA and Protein Markers in Formalin-Fixed Paraffin-Embedded Clinical Specimens of Pancreatic Cancer.

Composite images were generated to depict expression of marker protein and miRNA in pancreatic cancer. miR-375, insulin and glucagon exhibited acinar and islet cell staining.

Codetection of microRNA and Protein Markers in Formalin-Fixed Paraffin-Embedded Clinical Specimens of Prostate Cancer.

Composite images were generated to depict expression of marker protein and miRNA in prostate cancer. CK8/18 predominantly stained stroma, whereas miR-141 and E-cadherin exhibited staining of epithelia and stroma.

### Example 15

### Protocol for Fluorescent Staining to Codetect Protein and miRNA Markers in Cancer Tissue Specimens

The following is the general protocol for automated co-staining of miR-155, CD68, CD3, and MPO (Myeloperoxidase) in a cancer tissue sample.

Clinical specimens were placed on a slide. The slide was heated to 65° C. for 30 minutes and subsequently allowed to cool to 26° C. for 1 minute. The sample was dewaxed for 5 minutes with xylene and the slide was washed 2×30 seconds with alcohol (ethanol), 1×30 seconds with DI water, and 3×30 seconds with IHC wash. Proteinase K (50 μL; 10 μg/ml) was applied to the sample, and the sample was incubated at 37° C. for 20 minutes. The sample was washed for seconds with xylene. The sample was subsequently incubated in a solution of 0.2% glycine (in PBS; 300 μL) for 1 minute and washed 3×30 seconds with IHC wash.

Fixation.

The sample was fixed with paraformaldehyde (300 μL) for 10 minutes, washed 3×30 seconds with IHC wash, incubated in acetic acid (300 μL) for 2 minutes, washed 2×30 seconds with IHC wash, incubated with TRITON X-100 in PBS (300 μL) for 5 minutes and washed again 3×30 seconds with IHC wash.

Probe Hybridization.

Prehybrization solution (50 μL) was added to sample and the sample was incubated at 45° C. for 15 minutes. Double FAM-tagged (5′ and 3′ ends) miR-155 probe (50 μL) was added to the sample, and the sample was incubated at 50° C. for 75 minutes. The sample was washed for 30 seconds in ISH wash and subsequently washed in SSC buffer for 5 minutes at 50° C., DEPC water for 30 seconds, ISH wash for 30 seconds, and DEPC water for 30 seconds. Hydrogen peroxide (300 μL) was added to the sample for 15 minutes to block endogenous horseradish peroxidase activity. The sample was again washed with ISH wash for 30 seconds, DEPC water for 30 seconds, and ISH wash for 30 seconds. The sample was subsequently blocked with BSA (300 μL) for 20 minutes followed by a 5-minute was in PBT.

Antibody Binding and TSA.

Anti-FITC primary antibody (50 μL) was incubated with the sample for 45 minutes at 37° C., and the sample was subsequently washed for 2 minutes each with DEPC water, ISH wash, and DEPC water. Subsequently, an anti-rabbit horseradish peroxidase (HRP)-conjugated antibody (50 μL; 1:500 dilution) was incubated with the sample for 30 minutes at 37° C. followed by consecutive washes in DEPC water (2 minutes), ISH wash (2 minutes), and DEPC water (2 minutes). TSA green substrate (50 μL; 1:200 dilution) was added and the sample was incubated for 20 minutes at 25° C. Again, the sample was subsequently washed in ISH wash for 2 minutes, DEPC water for 2 minutes, and ISH wash for 2 minutes. Hydrogen peroxide (300 μL) was added to the sample for 15 minutes and the sample was washed consecutively for 30 seconds in each of DEPC water, ISH wash, and DEPC water. PBT (300 μL) was added to the sample for 5 minutes followed by a 45 minute incubation in streptavidin-conjugated HRP (50 μL) at 37° C. The sample was washed for 2 minutes in each of ISH wash, DEPC water, and ISH wash and TSA red substrate (50 μL; 1:500 dilution) was added for 15 minutes at 25° C. The sample was again washed for 2 minutes in DEPC water, 2 minutes in ISH wash and 2 minutes in DEPC water. The sample was subjected to HEIR at 90° C. for 10 minutes and washed briefly and consecutively in ISH wash, DEPC water, and ISH wash. Hydrogen peroxide (300 μL) was again added to the sample for 15 minutes and the sample was washed consecutively for 30 seconds in each of ISH wash, DEPC water, and ISH wash. The sample was incubated with BSA (300 μL) for 20 minutes and washed with PBT for 5 minutes. Primary anti-CD68 IgG1 antibody (50 μL) was incubated with the sample for 30 minutes at 37° C. Subsequently, the sample was washed for 2 minutes in DEPC water, 2 minutes in ISH wash, and 2 minutes in DEPC water. Secondary anti-IgG1 isotype-specific HRP-conjugated antibody was added to the sample and the sample was incubated at 37° C. for 30 minutes. The sample was briefly washed in ISH wash and DEPC water. TSA blue substrate (50 μL; 1:500 dilution) was added for 15 minutes at 25° C. and the sample was washed for 2 minutes in DEPC water, 2 minutes in ISH wash and 2 minutes in DEPC water. Hydrogen peroxide (300 μL) was added to the sample for 15 minutes and the sample was washed consecutively for 30 seconds in each of ISH wash, DEPC water, and ISH wash. The sample was washed with PBT for 5 minutes and anti-CD3 IgG2 antibody (50 μL) was added to the sample for 30 minutes at 37° C. The sample was washed for 2 minutes in DEPC water, 2 minutes in ISH wash, and 2 minutes in DEPC water. Secondary anti-IgG2a isotype-specific antibody (50 μL) was incubated with the sample for 30 minutes at 37° C., and the sample was subsequently washed for 20 seconds in each of ISH wash, DEPC water, and ISH wash. TSA daylight 594 substrate (50 μL; 1:100 dilution) was added to the sample and the sample was incubated at 25° C. for 15 minutes. Hydrogen peroxide (300 μL) was added to the sample for 15 minutes and the sample was washed consecutively for 30 seconds in each of ISH wash, DEPC water, and ISH wash. The sample was washed with PBT for 5 minutes and anti-MPO rabbit antibody (50 μL) was incubated with the sample for 30 minutes at 37° C. The sample was subsequently washed for 20 seconds in each of DEPC water, ISH wash, and DEPC water. The sample was incubated for 30 minutes at 37° C. with anti-rabbit HRP-conjugated secondary antibody and subsequently washed for 20 seconds in each of ISH wash, DEPC water, and ISH wash. TSA daylight 680 substrate (50 μL) was incubated with the sample for 15 minutes at 25° C., and the sample was briefly washed and analyzed.

### Example 16

### Protocol for Combination of Chromogenic and Fluorescent Staining to Codetect Protein and miRNA Markers in Cancer Tissue Specimens

The following is the general protocol for automated co-staining of miR-21, CK19, and HER2 in a cancer tissue sample.

Clinical specimens were placed on a slide. The slide was heated to 65° C. for 30 minutes and subsequently allowed to cool to 26° C. for 1 minute. The sample was dewaxed for 5 minutes with xylene and the slide was washed 2×30 seconds with alcohol (ethanol), 1×30 seconds with DI water, and 3×30 seconds with IHC wash. Proteinase K (50 μL; 10 μg/ml) was applied to the sample, and the sample was incubated at 37° C. for 20 minutes. The sample was washed for seconds with xylene. The sample was subsequently incubated in a solution of 0.2% glycine (in PBS; 300 μL) for 1 minute and washed 3×30 seconds with IHC wash.

Fixation.

The sample was fixed with paraformaldehyde (300 μL) for 10 minutes, washed 3×30 seconds with IHC wash, incubated in acetic acid (300 μL) for 2 minutes, washed 2×30 seconds with IHC wash, incubated with TRITON X-100 in PBS (300 μL) for 5 minutes and washed again 3×30 seconds with IHC wash.

Probe Hybridization.

Prehybrization solution (50 μL) was added to sample and the sample was incubated at 45° C. for 15 minutes. Labeled miR-21 probe (50 μL) was added to the sample, and the sample was incubated at 45° C. for 75 minutes. The sample was washed for 30 seconds in ISH wash and subsequently washed in SSC buffer for 5 minutes at 45° C., DEPC water for 30 seconds, ISH wash for 30 seconds, and DEPC water for 30 seconds. Hydrogen peroxide (300 μL) was added to the sample for 15 minutes to block endogenous horseradish peroxidase activity. The sample was again washed with ISH wash for 30 seconds, DEPC water for 30 seconds, and ISH wash for 30 seconds. The sample was subsequently blocked with BSA (300 μL) for 20 minutes followed by a 5-minute was in PBT.

Antibody Binding and TSA.

Anti-FITC primary antibody (50 μL) was incubated with the sample for 45 minutes at 37° C., and the sample was subsequently washed for 2 minutes each with DEPC water, ISH wash, and DEPC water. Subsequently, an anti-rabbit horseradish peroxidase (HRP)-conjugated antibody (50 μL; 1:500 dilution) was incubated with the sample for 30 minutes at 37° C. followed by consecutive washes in DEPC water (2 minutes), ISH wash (2 minutes), and DEPC water (2 minutes). TSA green substrate (50 μL; 1:200 dilution) was added and the sample was incubated for 20 minutes at 25° C. Again, the sample was subsequently washed in ISH wash for 2 minutes, DEPC water for 2 minutes, and ISH wash for 2 minutes. Hydrogen peroxide (300 μL) was added to the sample for 15 minutes and the sample was washed consecutively for 30 seconds in each of DEPC water, ISH wash, and DEPC water. PBT (300 μL) was added to the sample for 5 minutes followed by a 45 minute incubation in streptavidin-conjugated HRP (50 μL) at 37° C. The sample was washed for 2 minutes in each of ISH wash, DEPC water, and ISH wash and TSA red substrate (50 μL; 1:500 dilution) was added for 15 minutes at 25° C. The sample was again washed for 2 minutes in DEPC water, 2 minutes in ISH wash and 2 minutes in DEPC water. Hydrogen peroxide (300 μL) was again added to the sample for 15 minutes and the sample was washed consecutively for 30 seconds in each of ISH wash, DEPC water, and ISH wash. The sample was washed with PBT for 5 minutes. Primary anti-CK19 antibody (50 μL; 1:200) was incubated with the sample for 30 minutes at 37° C. Subsequently, the sample was washed for minutes in DEPC water, 2 minutes in ISH wash, and 2 minutes in DEPC water. Secondary anti-mouse HRP-conjugated antibody (1:500) was added to the sample and the sample was incubated at 37° C. for 30 minutes. The sample was briefly washed in ISH wash and DEPC water. TSA blue substrate (50 μL; 1:500 dilution) was added for 15 minutes at 25° C. and the sample was washed for 2 minutes in DEPC water, 2 minutes in ISH wash and 2 minutes in DEPC water. The sample was subjected to HEIR at 90° C. for 10 minutes and washed briefly and consecutively in ISH wash, DEPC water, and ISH wash. Hydrogen peroxide (300 μL) was again added to the sample for 15 minutes and the sample was washed consecutively for 30 seconds in each of ISH wash, DEPC water, and ISH wash. The sample was incubated with BSA (300 μL) for 20 minutes. The sample was washed with PBT for 5 minutes and anti-Her2 mouse antibody and anti-Pten antibody (50 μL) were added to the sample for 30 minutes at 37° C. The sample was washed for 2 minutes in DEPC water, 2 minutes in ISH wash, and 2 minutes in DEPC water. Secondary anti-mouse HRP-conjugated antibody and anti-rabbit AP-conjugated antibody (50 μL) were incubated with the sample for 30 minutes at 37° C., and the sample was subsequently washed for 20 seconds in each of ISH wash, DEPC water, and ISH wash. DAB substrate (50 μL) was added to the sample and the sample was incubated at 25° C. for 20 minutes. The sample was washed consecutively for 20 seconds in each of ISH wash, DEPC water, and ISH wash. The sample was subsequently incubated in BCIP-NBT solution (50 μL) for 20 minutes at 25° C. The sample was washed with ISH wash and DEPC and incubated with Fast Red substrate for 3 minutes. The sample was subsequently washed for 3×2 minutes and 20 seconds in DI water. PROLONG with DAPI gold was added to the sample, and the sample was analyzed.

What is claimed is:

1. A method for the codetection of microRNA and protein in a biological sample comprising
(a) contacting a biological sample with a probe that binds to a microRNA,
(b) detecting binding of the probe to the microRNA with a label,
(c) inactivating the label,
(d) contacting the biological sample with a binding agent that specifically binds to a protein, and
(e) detecting binding of the binding agent to the protein, thereby codetecting the microRNA and protein in the biological sample.

2. The method of claim 1, wherein the label comprises at least one hapten, an anti-hapten antibody, and an enzyme-conjugated secondary antibody specific for the anti-hapten antibody.

3. The method of claim 2, wherein the enzyme of the enzyme-conjugated secondary antibody is horseradish peroxidase.

4. The method of claim 1, wherein the probe is selected from the group of SEQ ID NO:1 to 56.

5. The method of claim 1, wherein the probe is a modified probe.

6. The method of claim 1, wherein the binding agent that specifically binds to the protein comprises an antibody.

7. The method of claim 6, wherein binding of the binding agent to the protein is detected by
(i) contacting the antibody with an enzyme-conjugated secondary antibody, and
(ii) detecting the enzyme activity.

8. The method of claim 7, wherein the enzyme of the enzyme-conjugated secondary antibody is horseradish peroxidase.

9. The method of claim 1, wherein the protein is a cell type-specific or functional marker selected from the group of amylase, cytokeratin (CK) 5/6, CK7, CK8/18, CK14, CK19, CK20, cluster of differentiation (CD)3, CD4, CD8, CD11b, CD11c, CD19, CD20, CD31, CD34, CD24, CD44, CD45, CD68, CD86, CD105, myeloperoxidase, E-cadherin, laminin, estrogen receptor (ER), Glucagon, Human Epidermal growth factor Receptor 2 (HER2), Insulin, Ki-67, phosphorylated v-akt murine thymoma viral oncogene (pAKT), protein 15 (p15), p16, p21, p27, p53, p63, mutS homolog 6 (MSH-6), Proliferating Cell Nuclear Antigen (PCNA), Cyclin D1, Cyclin E, Progesterone Receptor (PR), Phosphatase and Tensin Homolog (PTEN), Smooth Muscle Actin, Tubulin, Vimentin and somatostatin.

10. The method of claim 1, wherein the biological sample is a biopsy sample.

11. The method of claim 1, further comprising detecting a non-coding RNA in the biological sample.

12. A method for the codetection of multiple microRNAs and proteins in a biological sample comprising
(a) contacting a biological sample with a probe that binds to a microRNA,
(b) detecting binding of the probe to the microRNA with a label,
(c) inactivating the label,
(d) contacting the biological sample with an antibody that specifically binds to a protein,
(e) detecting binding of the antibody to the protein with a secondary antibody reagent,
(f) inactivating the secondary antibody reagent, and
(g) repeating steps (a) to (f) for the codetection of multiple microRNAs and proteins in the biological sample.

13. A kit comprising
(a) a probe that binds to a microRNA, wherein the probe is selected from the group of SEQ ID NO:1 to 56;
(b) a binding agent that specifically binds to a protein; and
(c) instructions for hybridizing the probe and binding agent to the microRNA and protein, respectively.


SEQUENCE LISTING (58 sequences)

SEQ ID NO: 1 - DNA, 22 bp, Artificial Sequence
  Synthetic oligonucleotide
aactatacaacctactacctca

SEQ ID NO: 2 - DNA, 22 bp, Artificial Sequence
  Synthetic oligonucleotide
tcaacatcagtctgataagcta

SEQ ID NO: 3 - DNA, 22 bp, Artificial Sequence
  Synthetic oligonucleotide
ctgttcctgctgaactgagcca

SEQ ID NO: 4 - DNA, 22 bp, Artificial Sequence
  Synthetic oligonucleotide
acaaccagctaagacactgcca

SEQ ID NO: 5 - DNA, 23 bp, Artificial Sequence
  Synthetic oligonucleotide
gcaatcagctaactacactgcct

SEQ ID NO: 6 - DNA, 22 bp, Artificial Sequence
  Synthetic oligonucleotide
tcacaagttagggtctcaggga

SEQ ID NO: 7 - DNA, 22 bp, Artificial Sequence
  Synthetic oligonucleotide
cgcattattactcacggtacga

SEQ ID NO: 8 - DNA, 22 bp, Artificial Sequence
  Synthetic oligonucleotide
ccatctttaccagacagtgtta

SEQ ID NO: 9 - DNA, 21 bp, Artificial Sequence
  Synthetic oligonucleotide
gagctacagtgcttcatctca

SEQ ID NO: 10 - DNA, 24 bp, Artificial Sequence
  Synthetic oligonucleotide
aagggattcctgggaaaactggac

SEQ ID NO: 11 - DNA, 23 bp, Artificial Sequence
  Synthetic oligonucleotide
ttaatgctaatcgtgataggggt

SEQ ID NO: 12 - DNA, 22 bp, Artificial Sequence
  Synthetic oligonucleotide
cccaacaacatgaaactaccta

SEQ ID NO: 13 - DNA, 23 bp, Artificial Sequence
  Synthetic oligonucleotide
gtcatcattaccaggcagtatta

SEQ ID NO: 14 - DNA, 22 bp, Artificial Sequence
  Synthetic oligonucleotide
cagactccggtggaatgaagga

SEQ ID NO: 15 - DNA, 21 bp, Artificial Sequence
  Synthetic oligonucleotide
ctgcctgtctgtgcctgctgt

SEQ ID NO: 16 - DNA, 23 bp, Artificial Sequence
  Synthetic oligonucleotide
gaaacccagcagacaatgtagct

SEQ ID NO: 17 - DNA, 22 bp, Artificial Sequence
  Synthetic oligonucleotide
tcacgcgagccgaacgaacaaa

SEQ ID NO: 18 - DNA, 23 bp, Artificial Sequence
  Synthetic oligonucleotide
aaactcagtaatggtaacggttt

SEQ ID NO: 19 - DNA, 23 bp, Artificial Sequence
  Synthetic oligonucleotide
cacaaattcggttctacagggta

SEQ ID NO: 20 - DNA, 22 bp, Artificial Sequence
  Synthetic oligonucleotide
aaccacacaacctactacctca

SEQ ID NO: 21 - DNA, 22 bp, Artificial Sequence
  Synthetic oligonucleotide
aactatacaatctactacctca

SEQ ID NO: 22 - DNA, 22 bp, Artificial Sequence
  Synthetic oligonucleotide
aactgtacaaactactacctca

SEQ ID NO: 23 - DNA, 23 bp, Artificial Sequence
  Synthetic oligonucleotide
acaacaaaatcactagtcttcca

SEQ ID NO: 24 - DNA, 22 bp, Artificial Sequence
  Synthetic oligonucleotide
tgtaaaccatgatgtgctgcta

SEQ ID NO: 25 - DNA, 23 bp, Artificial Sequence
  Synthetic oligonucleotide
ctacctgcactgtaagcactttg

SEQ ID NO: 26 - DNA, 23 bp, Artificial Sequence
  Synthetic oligonucleotide
ctatctgcactagatgcacctta

SEQ ID NO: 27 - DNA, 21 bp, Artificial Sequence
  Synthetic oligonucleotide
gcagaacttagccactgtgaa

SEQ ID NO: 28 - DNA, 23 bp, Artificial Sequence
  Synthetic oligonucleotide
aacactgatttcaaatggtgcta

SEQ ID NO: 29 - DNA, 22 bp, Artificial Sequence
  Synthetic oligonucleotide
cttccagtcgaggatgtttaca

SEQ ID NO: 30 - DNA, 21 bp, Artificial Sequence
  Synthetic oligonucleotide
agctatgccagcatcttgcct

SEQ ID NO: 31 - DNA, 22 bp, Artificial Sequence
  Synthetic oligonucleotide
acaggccgggacaagtgcaata

SEQ ID NO: 32 - DNA, 22 bp, Artificial Sequence
  Synthetic oligonucleotide
cacaagttcggatctacgggtt

SEQ ID NO: 33 - DNA, 21 bp, Artificial Sequence
  Synthetic oligonucleotide
atctgcactgtcagcacttta

SEQ ID NO: 34 - DNA, 23 bp, Artificial Sequence
  Synthetic oligonucleotide
tgatagccctgtacaatgctgct

SEQ ID NO: 35 - DNA, 22 bp, Artificial Sequence
  Synthetic oligonucleotide
caaacaccattgtcacactcca

SEQ ID NO: 36 - DNA, 20 bp, Artificial Sequence
  Synthetic oligonucleotide
ggcattcaccgcgtgcctta

SEQ ID NO: 37 - DNA, 21 bp, Artificial Sequence
  Synthetic oligonucleotide
agtagtgctttctactttatg

SEQ ID NO: 38 - DNA, 22 bp, Artificial Sequence
  Synthetic oligonucleotide
aacccatggaattcagttctca

SEQ ID NO: 39 - DNA, 22 bp, Artificial Sequence
  Synthetic oligonucleotide
acaaagttctgtagtgcactga

SEQ ID NO: 40 - DNA, 23 bp, Artificial Sequence
  Synthetic oligonucleotide
actcaccgacagcgttgaatgtt

SEQ ID NO: 41 - DNA, 23 bp, Artificial Sequence
  Synthetic oligonucleotide
acccaccgacagcaatgaatgtt

SEQ ID NO: 42 - DNA, 23 bp, Artificial Sequence
  Synthetic oligonucleotide
cagctgcttttgggattccgttg

SEQ ID NO: 43 - DNA, 22 bp, Artificial Sequence
  Synthetic oligonucleotide
cccaacaacaggaaactaccta

SEQ ID NO: 44 - DNA, 23 bp, Artificial Sequence
  Synthetic oligonucleotide
tccatcattacccggcagtatta

SEQ ID NO: 45 - DNA, 22 bp, Artificial Sequence
  Synthetic oligonucleotide
ctagtggtcctaaacatttcac

SEQ ID NO: 46 - DNA, 22 bp, Artificial Sequence
  Synthetic oligonucleotide
aggcataggatgacaaagggaa

SEQ ID NO: 47 - DNA, 22 bp, Artificial Sequence
  Synthetic oligonucleotide
ccacacacttccttacattcca

SEQ ID NO: 48 - DNA, 22 bp, Artificial Sequence
  Synthetic oligonucleotide
tcagccgctgtcacacgcacag

SEQ ID NO: 49 - DNA, 22 bp, Artificial Sequence
  Synthetic oligonucleotide
tcacagttgccagctgagatta

SEQ ID NO: 50 - DNA, 23 bp, Artificial Sequence
  Synthetic oligonucleotide
tccaatcagttcctgatgcagta

SEQ ID NO: 51 - DNA, 21 bp, Artificial Sequence
  Synthetic oligonucleotide
acccagtagccagatgtagct

SEQ ID NO: 52 - DNA, 23 bp, Artificial Sequence
  Synthetic oligonucleotide
acatttttcgttattgctcttga

SEQ ID NO: 53 - DNA, 23 bp, Artificial Sequence
  Synthetic oligonucleotide
acaccccaaaatcgaagcacttc

SEQ ID NO: 54 - DNA, 21 bp, Artificial Sequence
  Synthetic oligonucleotide
acgtggattttcctctatgat

SEQ ID NO: 55 - DNA, 22 bp, Artificial Sequence
  Synthetic oligonucleotide
cagaaagtgcttccctctagag

SEQ ID NO: 56 - DNA, 23 bp, Artificial Sequence
  Synthetic oligonucleotide
tccatcatcaaaacaaatggagt

SEQ ID NO: 57 - DNA, 40 bp, Artificial Sequence
  Synthetic oligonucleotide
cgtgtcatccttgcgcaggggccatgctaatcttctctgt

SEQ ID NO: 58 - DNA, 35 bp, Artificial Sequence
  Synthetic oligonucleotide
gggcagacgttcgaatgggtcgtcgccgccacggg